SEQUENCE LISTING (26 sequences)

SEQ ID NO: 1 - protein, 500 aa, synthetic construct [sequence omitted]

SEQ ID NO: 2 - DNA, 53 bp, synthetic construct
  source 1..53: mol_type = other DNA
gttgcgcagccatcgacgtcagacgcggaagcttcgatcaactacgcagacag

SEQ ID NO: 3 - DNA, 104 bp, synthetic construct
  source 1..104: mol_type = other DNA
gttgtggtttgtctggtcaaccaccgcggactccgtcgtcaggatcattcagttgcgcagccatcgacgtcagacgcggaagcttcgatcaactacgcagacag

SEQ ID NO: 4 - DNA, 101 bp, synthetic construct
  source 1..101: mol_type = other DNA
gttgcgcagccatcgacgtcagtggtttgtctggtcaaccaccgcggactccgtcgtcaggatcattcagacgcggaagcttcgatcaactacgcagacag

SEQ ID NO: 5 - DNA, 98 bp, synthetic construct
  source 1..98: mol_type = other DNA
gttgcgcagccatcgacgtcagacgcggaagcttcggtggtttgtctggtcaaccaccgcggactccgtcgtcaggatcatatcaactacgcagacag

SEQ ID NO: 6 - DNA, 1660 bp, synthetic construct
  source 1..1660: mol_type = other DNA
ggcagaacccctttgattttggatccggtgagggcagaggaagtctactaacatgcggtgacgtggaggagaatccgggccctatgccaaaaaagaaaagaaaagtgtatccctatgatgtccccgattatgccggttcaagagccctggtcgtgattagactgagccgagtgacagacgccaccacaagtcccgagagacagctggaatcatgccagcagctctgtgctcagcggggttgggatgtggtcggcgtggcagaggatctggacgtgagcggggccgtcgatccattcgacagaaagaggaggcccaacctggcaagatggctcgctttcgaggaacagccctttgatgtgatcgtcgcctacagagtggaccggctgacccgctcaattcgacatctccagcagctggtgcattgggctgaggaccacaagaaactggtggtcagcgcaacagaagcccacttcgatactaccacaccttttgccgctgtggtcatcgcactgatgggcactgtggcccagatggagctcgaagctatcaaggagcgaaacaggagcgcagcccatttcaatattagggccggtaaatacagaggctccctgcccccttggggatatctccctaccagggtggatggggagtggagactggtgccagaccccgtccagagagagcggattctggaagtgtaccacagagtggtcgataaccacgaaccactccatctggtggcacacgacctgaatagacgcggcgtgctctctccaaaggattattttgctcagctgcagggaagagagccacagggaagagaatggagtgctactgcactgaagagatctatgatcagtgaggctatgctgggttacgcaacactcaatggcaaaactgtccgggacgatgacggagcccctctggtgagggctgagcctattctcaccagagagcagctcgaagctctgcgggcagaactggtcaagactagtcgcgccaaacctgccgtgagcaccccaagcctgctcctgagggtgctgttctgcgccgtctgtggagagccagcatacaagtttgccggcggagggcgcaaacatccccgctatcgatgcaggagcatggggttccctaagcactgtggaaacgggacagtggccatggctgagtgggacgccttttgcgaggaacaggtgctggatctcctgggtgacgctgagcggctggaaaaagtgtgggtggcaggatctgactccgctgtggagctggcagaagtcaatgccgagctcgtggatctgacttccctcatcggatctcctgcatatagagctgggtccccacagagagaagctctggacgcacgaattgctgcactcgctgctagacaggaggaactggagggcctggaggccaggccctctggatgggagtggcgagaaaccggacagaggtttggggattggtggagggagcaggacaccgcagccaagaacacatggctgagatccatgaatgtccggctcacattcgacgtgcgcggtggcctgactcgaaccatcgattttggcgacctgcaggagtatgaacagcacctgagactggggtccgtggtcgaaagactgcacactgggatgtcctaaacccttgccgtctgcgccgt

SEQ ID NO: 7 - DNA, 989 bp, synthetic construct
  source 1..989: mol_type = other DNA
gttgcgcagccatcgacgtcagtggtttgtctggtcaaccaccgcggactcagtggtgtacggtacaaacccattaattaaggccttaattaggctgcgcgaagcttgcagatctgcgactctagaggatctgcgactctagaggatcataatcagccataccacattttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcccgagcgcgcagccttaattaaggcttgtcgacgacggcggactccgtcgtcaggatcattcagacgcggaagcttcgatcaactacgcggacag

SEQ ID NO: 8 - DNA, 2189 bp, synthetic construct
  source 1..2189: mol_type = other DNA
agaacccctttgatttttaatgcatcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatgccaaaaaagaaaagaaaagtgtatccctatgatgtccccgattatgccggttcaagagccctggtcgtgattagactgagccgagtgacagacgccaccacaagtcccgagagacagctggaatcatgccagcagctctgtgctcagcggggttgggatgtggtcggcgtggcagaggatctggacgtgagcggggccgtcgatccattcgacagaaagaggaggcccaacctggcaagatggctcgctttcgaggaacagccctttgatgtgatcgtcgcctacagagtggaccggctgacccgctcaattcgacatctccagcagctggtgcattgggctgaggaccacaagaaactggtggtcagcgcaacagaagcccacttcgatactaccacaccttttgccgctgtggtcatcgcactgatgggcactgtggcccagatggagctcgaagctatcaaggagcgaaacaggagcgcagcccatttcaatattagggccggtaaatacagaggctccctgcccccttggggatatctccctaccagggtggatggggagtggagactggtgccagaccccgtccagagagagcggattctggaagtgtaccacagagtggtcgataaccacgaaccactccatctggtggcacacgacctgaatagacgcggcgtgctctctccaaaggattattttgctcagctgcagggaagagagccacagggaagagaatggagtgctactgcactgaagagatctatgatcagtgaggctatgctgggttacgcaacactcaatggcaaaactgtccgggacgatgacggagcccctctggtgagggctgagcctattctcaccagagagcagctcgaagctctgcgggcagaactggtcaagactagtcgcgccaaacctgccgtgagcaccccaagcctgctcctgagggtgctgttctgcgccgtctgtggagagccagcatacaagtttgccggcggagggcgcaaacatccccgctatcgatgcaggagcatggggttccctaagcactgtggaaacgggacagtggccatggctgagtgggacgccttttgcgaggaacaggtgctggatctcctgggtgacgctgagcggctggaaaaagtgtgggtggcaggatctgactccgctgtggagctggcagaagtcaatgccgagctcgtggatctgacttccctcatcggatctcctgcatatagagctgggtccccacagagagaagctctggacgcacgaattgctgcactcgctgctagacaggaggaactggagggcctggaggccaggccctctggatgggagtggcgagaaaccggacagaggtttggggattggtggagggagcaggacaccgcagccaagaacacatggctgagatccatgaatgtccggctcacattcgacgtgcgcggtggcctgactcgaaccatcgattttggcgacctgcaggagtatgaacagcacctgagactggggtccgtggtcgaaagactgcacactgggatgtcctaaacccttgccgtctgcgccgt

SEQ ID NO: 9 - DNA, 2490 bp, synthetic construct
  source 1..2490: mol_type = other DNA
gttgcgcagccatcgacgtcagtggtttgtctggtcaaccaccgcggactcagtggtgtacggtacaaacccattaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccctagcgtttaaacgggccctctagactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaaggcttgtcgacgacggcggactccgtcgtcaggatcattcagacgcggaagcttcgatcaactacgcagacag

SEQ ID NO: 10 - DNA, 4429 bp, synthetic construct
  source 1..4429: mol_type = other DNA
ctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacaatcaagcttctgacctcttctcttcctcccacagggcctcgagagatctggcagcggagcaacaaacttctcactactcaaacaagcaggtgacgtggaggagaatcccgggcctaggctcgagatgccaaaaaagaaaagaaaagtgtatccctatgatgtccccgattatgccggttcaagagccctggtcgtgattagactgagccgagtgacagacgccaccacaagtcccgagagacagctggaatcatgccagcagctctgtgctcagcggggttgggatgtggtcggcgtggcagaggatctggacgtgagcggggccgtcgatccattcgacagaaagaggaggcccaacctggcaagatggctcgctttcgaggaacagccctttgatgtgatcgtcgcctacagagtggaccggctgacccgctcaattcgacatctccagcagctggtgcattgggctgaggaccacaagaaactggtggtcagcgcaacagaagcccacttcgatactaccacaccttttgccgctgtggtcatcgcactgatgggcactgtggcccagatggagctcgaagctatcaaggagcgaaacaggagcgcagcccatttcaatattagggccggtaaatacagaggctccctgcccccttggggatatctccctaccagggtggatggggagtggagactggtgccagaccccgtccagagagagcggattctggaagtgtaccacagagtggtcgataaccacgaaccactccatctggtggcacacgacctgaatagacgcggcgtgctctctccaaaggattattttgctcagctgcagggaagagagccacagggaagagaatggagtgctactgcactgaagagatctatgatcagtgaggctatgctgggttacgcaacactcaatggcaaaactgtccgggacgatgacggagcccctctggtgagggctgagcctattctcaccagagagcagctcgaagctctgcgggcagaactggtcaagactagtcgcgccaaacctgccgtgagcaccccaagcctgctcctgagggtgctgttctgcgccgtctgtggagagccagcatacaagtttgccggcggagggcgcaaacatccccgctatcgatgcaggagcatggggttccctaagcactgtggaaacgggacagtggccatggctgagtgggacgccttttgcgaggaacaggtgctggatctcctgggtgacgctgagcggctggaaaaagtgtgggtggcaggatctgactccgctgtggagctggcagaagtcaatgccgagctcgtggatctgacttccctcatcggatctcctgcatatagagctgggtccccacagagagaagctctggacgcacgaattgctgcactcgctgctagacaggaggaactggagggcctggaggccaggccctctggatgggagtggcgagaaaccggacagaggtttggggattggtggagggagcaggacaccgcagccaagaacacatggctgagatccatgaatgtccggctcacattcgacgtgcgcggtggcctgactcgaaccatcgattttggcgacctgcaggagtatgaacagcacctgagactggggtccgtggtcgaaagactgcacactgggatgtccggatccggtgagggcagaggaagtctactaacatgcggtgacgtggaggagaatccgggccctgaattcgccaccatgacagaatacaagcccacagtcaggcttgcaactagagatgacgttcccagagcagtgagaaccctggcagctgcttttgcagactatccggccacgaggcacactgtcgatcccgatcggcacatcgagcgcgttacagaattgcaggaactgttcctgacaagagttgggctcgacattggtaaagtgtgggtcgctgacgacggggcagctgttgcggtgtggaccacaccggagagtgtggaggccggtgctgtgtttgccgaaattggtccacgcatggccgaactctccggatctcggttggccgcacagcagcagatggaaggcctgctggcgcctcaccgacctaaagagcctgcatggtttctggccaccgtcggcgtatcccccgatcatcagggtaagggcctcggcagcgccgtcgtgctgccgggtgttgaggcagctgaaagagcaggcgtgccggcgtttttggaaacaagtgcaccgaggaatctcccattttacgagagactggggttcaccgtgacagccgatgtcgaagtgcccgaaggccccaggacctggtgtatgacccgcaagcccggtgcctaggatccgatctttttccctctgccaaaaattatggggacatcatgaagccccttgagcatctgacttctggctaataaaggaaatttattttcattgcaatagtgtgttggaattttttgtgtctctcactcgtcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagac

SEQ ID NO: 11 - DNA, 45 bp, synthetic construct
  source 1..45: mol_type = other DNA
gtggtttgtctggtcaaccaccgcggactccgtcgtcaggatcat

SEQ ID NO: 12 - DNA, 21 bp, synthetic construct
  source 1..21: mol_type = other DNA
gataaatctggagccggtgag

SEQ ID NO: 13 - DNA, 22 bp, synthetic construct
  source 1..22: mol_type = other DNA
agataactacgatacgggaggg

SEQ ID NO: 14 - DNA, 23 bp, synthetic construct
  source 1..23: mol_type = other DNA
tgggtctcgcggtatcattgcag

SEQ ID NO: 15 - DNA, 20 bp, synthetic construct
  source 1..20: mol_type = other DNA
ctgggctcttcctcttcctc

SEQ ID NO: 16 - DNA, 20 bp, synthetic construct
  source 1..20: mol_type = other DNA
gatgtggcgctacaaatggt

SEQ ID NO: 17 - DNA, 20 bp, synthetic construct
  source 1..20: mol_type = other DNA
ttcagccgccgcactgtgcg

SEQ ID NO: 18 - DNA, 20 bp, synthetic construct
  source 1..20: mol_type = other DNA
aagacctcgcacgtaactca

SEQ ID NO: 19 - DNA, 20 bp, synthetic construct
  source 1..20: mol_type = other DNA
ggcatgacactacgaccaac

SEQ ID NO: 20 - DNA, 21 bp, synthetic construct
  source 1..21: mol_type = other DNA
cggttgtctcggcgcactccg

SEQ ID NO: 21 - DNA, 20 bp, synthetic construct
  source 1..20: mol_type = other DNA
ctcaacgaccttcgaacacc

SEQ ID NO: 22 - DNA, 20 bp, synthetic construct
  source 1..20: mol_type = other DNA
acctcaaccacgtgatcctt

SEQ ID NO: 23 - DNA, 20 bp, synthetic construct
  source 1..20: mol_type = other DNA
ccggtcttgcaacggctgct

SEQ ID NO: 24 - DNA, 20 bp, synthetic construct
  source 1..20: mol_type = other DNA
atggacaagtggccacaaac

SEQ ID NO: 25 - DNA, 20 bp, synthetic construct
  source 1..20: mol_type = other DNA
atcagcggagaagggtgaaa

SEQ ID NO: 26 - DNA, 20 bp, synthetic construct
  source 1..20: mol_type = other DNA
cagccggtctgcgcctgtgc

(19) United States
(12) Patent Application Publication
(10) Pub. No.: US 2025/0320524 A1
(43) Pub. Date: Oct. 16, 2025

(54) EFFECTIVELY PACKAGING HIGH-QUALITY RAAV VECTORS BY MINICIRCLE DUAL TRANSFECTION

(71) Applicant: University of Massachusetts, Westborough, MA (US)

(72) Inventors: Dan Wang, Worcester, MA (US); Hao Liu, Worcester, MA (US); Guangping Gao, Worcester, MA (US)

(73) Assignee: University of Massachusetts, Westborough, MA (US)

(21) Appl. No.: 19/176,384

(22) Filed: Apr. 11, 2025

Related U.S. Application Data
(60) Provisional application No. 63/633,972, filed on Apr. 15, 2024.

(51) Int. Cl. C12N 15/86

(52) U.S. Cl. CPC C12N 15/86; C12N 2750/14122; C12N 2750/14143; C12N 2750/14152

(57) ABSTRACT

Aspects of the disclosure relate to methods for producing recombinant adeno-associated virus (rAAV) particles. The disclosure is based, in part, on minicircle DNA dual-transfection, which in some embodiments, significantly reduces plasmid consumption, vector impurity, and empty capsid formation, while at the same time generates a comparable or higher rAAV titer.

### RELATED APPLICATIONS

This application claims the benefit under 35 U.S.C. 119 (e) of the filing date of U.S. provisional application Ser. Nos. 63/633,972, filed Apr. 15, 2024, entitled “EFFECTIVELY PACKAGING HIGH-QUALITY RAAV VECTORS BY MINICIRCLE DUAL TRANSFECTION”, the entire contents of which are incorporated by reference herein.

### REFERENCE TO AN ELECTRONIC SEQUENCE LISTING

The contents of the electronic sequence listing (U012070196US01-SEQ-KZM.xml; Size: 36,202 bytes; and Date of Creation: Apr. 8, 2025) is herein incorporated by reference in its entirety.

### BACKGROUND OF INVENTION

Recombinant adeno-associated virus (rAAV) vectors are the leading platform for human gene therapy delivery. In addition, rAAV is widely used for functional genomic studies in biomedical research, such as molecular genetics and cancer biology. Production of high-quality rAAV packaging a desired transgene holds the key in both clinical application and basic research.

### SUMMARY OF INVENTION

Aspects of this disclosure relate to methods of minicircle dual transfection (e.g., methods of producing rAAVs with reduced plasmid cost, reduced plasmid backbone contamination, and a decrease in empty capsid formation). In some embodiments, minicircle dual transfection is also referred to as AAVPure^{Mfg}. The inventors discovered that culturing a host cell and introducing (i) a nucleic acid comprising a sequence encoding one or more adenovirus helper proteins and a sequence encoding a recombinase (pHelper-recombinase); and (ii) a nucleic acid comprising a transgene and a sequence encoding Rep and/or Cap proteins (pTrans/Cis) improved rAAV production. Methods of the disclosure Methods of the disclosure maintain high yield of rAAV production and enable efficient packaging of transgenes.

Accordingly, in some aspects, the disclosure provides a circular nucleic acid comprising a first nucleic acid sequence encoding one or more adenoviral helper factors; and a second nucleic acid sequence encoding a recombinase.

In some aspects, the disclosure provides a circular nucleic acid comprising a first nucleic acid sequence encoding an adeno-associated virus (AAV) Rep; a second nucleic acid sequence encoding a transgene comprising a promoter operably linked to a nucleic acid sequence encoding a gene product, flanked by AAV inverted terminal repeats (ITRs); and a third nucleic acid sequence encoding an AAV capsid protein.

In some aspects, the disclosure provides an rAAV production system comprising a first circular nucleic acid comprising a first nucleic acid sequence encoding one or more adenoviral helper factors; and a second nucleic acid sequence encoding a recombinase; and a second circular nucleic acid comprising a first nucleic acid sequence encoding an adeno-associated virus (AAV) Rep; a second nucleic acid sequence encoding a transgene comprising a promoter operably linked to a nucleic acid sequence encoding a gene product, flanked by AAV inverted terminal repeats (ITRs); and a third nucleic acid sequence encoding an AAV capsid protein.

In some embodiments, a circular nucleic acid comprises a plasmid. In some embodiments, a circular nucleic acid comprises a minicircle.

In some embodiments, a first nucleic acid sequence encoding one or more adenoviral helper factors is positioned 5′ relative to a nucleic acid sequence encoding a recombinase. In some embodiments, one or more adenoviral helper factors comprise E2A, E1B55K, E4orf6, and/or VA RNA transcription units.

In some embodiments, a recombinase is a Bxb1 recombinase. In some embodiments, a nucleic acid sequence encoding a recombinase is inserted before a stop codon of a nucleic acid sequence encoding one or more adenoviral helper factors. In some embodiments, a stop codon is a stop codon of E2A helper factor.

In some embodiments, a first recombinase recognition site is positioned between a first nucleic acid sequence and a second nucleic acid sequence; and a second recombinase recognition site positioned between a second nucleic acid sequence and a third nucleic acid sequence.

In some embodiments, a first recombinase recognition site comprises an attP site and a second recombinase recognition site comprises an attB site.

In some embodiments, a nucleic acid sequence is positioned between a p40 promoter of a first nucleic acid sequence and an intron of a third nucleic acid sequence.

In some embodiments, an intron of a third nucleic acid sequence is positioned upstream of a start codon of a nucleic acid sequence encoding an AAV capsid protein.

In some embodiments, a first nucleic acid sequence and second nucleic acid sequence are in a first open reading frame, and a third nucleic acid sequence is in a second open reading frame.

In some aspects, the disclosure provides a circular nucleic acid comprising a transgene comprising a promoter operably linked to a nucleic acid sequence encoding a gene product, flanked by AAV inverted terminal repeats (ITRs), and an attL recombinase recognition site positioned between the AAV ITRs.

In some embodiments, a circular nucleic acid lacks bacteria-derived DNA.

In some aspects, the disclosure provides a method for producing adeno-associated virus (rAAV) particles the method comprising introducing into a host cell a first circular nucleic acid comprising a first nucleic acid sequence encoding one or more adenoviral helper factors; and a second nucleic acid sequence encoding a recombinase; and a second circular nucleic acid comprising a first nucleic acid sequence encoding an adeno-associated virus (AAV) Rep; a second nucleic acid sequence encoding a transgene comprising a promoter operably linked to a nucleic acid sequence encoding a gene product, flanked by AAV inverted terminal repeats (ITRs); and a third nucleic acid sequence encoding an AAV capsid protein and culturing the host cell under conditions under which rAAV particles are produced.

In some embodiments, less than 1% of the rAAV particles comprise plasmid backbone DNA.

In some aspects, the disclosure provides a method for producing adeno-associated virus (rAAV) particles the method comprising introducing into a host cell: a first circular nucleic acid comprising a first nucleic acid sequence encoding one or more adenoviral helper factors; and a second nucleic acid sequence encoding a recombinase; and a second circular nucleic acid comprising a first nucleic acid sequence encoding an adeno-associated virus (AAV) Rep; a second nucleic acid sequence encoding a transgene comprising a promoter operably linked to a nucleic acid sequence encoding a gene product, flanked by AAV inverted terminal repeats (ITRs); a third nucleic acid sequence encoding an AAV capsid protein; a third circular nucleic acid sequence comprising a first nucleic acid sequence encoding one or more adenoviral helper factors, and culturing the host cell under conditions under which rAAV particles are produced. In some embodiments, the one or more adenoviral helper factors are E2A, E1B55K, E4orf6, and/or VA RNA transcription units (e.g., AdDeltaF6 plasmid). In some embodiments, less than 1% of the rAAV particles comprise plasmid backbone DNA.

Accordingly, in some aspects, the disclosure provides a host cell, wherein a recombinase is integrated into the genome of the host cell. In some embodiments, the recombinase is Bxb1. In some embodiments, the recombinase is integrated into a AAVS1 safe harbor locus of the host cell. In some embodiments, the host cell is a HEK293 cell.

In some aspects, the disclosure provides a method for producing adeno-associated virus (rAAV) particles the method comprising introducing into a host cell, wherein the host cell comprises a recombinase integrated into its genome: a first circular nucleic acid comprising a first nucleic acid sequence encoding an adeno-associated virus (AAV) Rep; a second nucleic acid sequence encoding a transgene comprising a promoter operably linked to a nucleic acid sequence encoding a gene product, flanked by AAV inverted terminal repeats (ITRs); a third nucleic acid sequence encoding an AAV capsid protein; a second circular nucleic acid sequence comprising a first nucleic acid sequence encoding E2A, E1B55K, E4orf6, and/or VA RNA transcription units, and culturing the host cell under conditions under which rAAV particles are produced.

### BRIEF DESCRIPTION OF DRAWINGS

FIGS. 1A-1B depict a schematic comparison between standard triple transfection and minicircle dual transfection. FIG. 1A depicts standard triple transfection, where three plasmids of pHelper, pTrans and pCis are co-transfected into a cell to generate desired rAAV. FIG. 1B depicts minicircle dual transfection, where two plasmids of pHelper-Bxb1 and pTrans/Cis are co-transfected into cells.

FIGS. 2A-2D show the identification of attR knock in site. FIG. 2A shows pTrans plasmid genomic structure. FIGS. 2B-2D show the packaging yield of EGFP by standard triple transfection with either traditional pTrans or engineered pTrans-attR with attR knock-in. The serotype is AAV2 (FIG. 2B), AAV5 (FIG. 2C) or AAV9 (FIG. 2D).

FIGS. 3A-3D show minicircle dual transfection produces comparable or higher titer of rAAV with reduced plasmid demand relative to standard triple transfection. FIGS. 3A-3C shows the packaging yield of EGFP in AAV2 (FIG. 3A), AAV5 (FIG. 3B), or AAV9 (FIG. 3C) by either standard triple transfection or minicircle dual transfection. FIG. 3D shows HEK293 cells transfected by either standard triple transfection or minicircle dual transfection using AAV2, AAV5, or AAV9.

FIGS. 4A-4C shows minicircle dual transfection generates rAAV with reduced plasmid backbone encapsidation relative to standard triple transfection. FIGS. 4A-4C show a 5′ plasmid backbone or 3′ plasmid backbone encapsidation ratio in ssAAV2.EGFP (FIG. 4A), ssAAV5.EGFP (FIG. 4B) or ssAAV9.EGFP (FIG. 4C) generated by either standard triple transfection or minicircle dual transfection

FIGS. 5A-5F shows minicircle dual transfection generates rAAV with reduced empty capsid formation relative to standard triple transfection. FIGS. 5A and 5D show the packaging yield of EGFP in AAV2 (FIG. 5A) and AAV9 (FIG. 5D) by either standard triple transfection or minicircle dual transfection. FIGS. 5B and 5E show AAV2 (FIG. 5B) and AAV9 (FIG. 5E) capsid titer determined by enzyme linked immunosorbent assay (ELISA). FIGS. 5C and 5F show the full capsid ratio, calculated as genome titer divided by capsid titer, in the crude lysate as described in FIG. 5A (FIG. 5C) or FIG. 5D (FIG. 5F), respectively.

FIGS. 6A-6E are schematics comparing triple transfection and AAVPure^{Mfg}. FIG. 6A depicts a schematic diagram illustrating how rAAV impurities, including plasmid backbone encapsidation and empty capsid, are generated in triple transfection. FIG. 6B depicts a schematic diagram illustrating how rAAV impurities, including plasmid backbone encapsidation and empty capsid, are mitigated in AAVPure^{Mfg}. FIG. 6C The gene structure of pTrans plasmid and the expressed Rep transcripts. attR is chosen to be inserted between P40 and intron, located at the 3′ region of Rep gene as shown in red. FIG. 6D shows simulated AAV2 Rep78 protein structure, with the region between P40 and intron highlighted in red. The attR insertion sites are indicated by white arrows. FIG. 6E depicts the packaging yield of AAV2.EGFP using unmodified pTrans (pRep2/Cap2) or modified variants (pRep2-attR/Cap2).

FIGS. 7A-7G show minicircle AAVPure^{Mfg} improves AAV2 vector purity. FIG. 7A depicts a schematic of the plasmid components in AAVPure^{Mfg} 1.0 and AAVPure^{Mfg} 1.1. FIG. 7B depicts a non-limiting example of the experimental procedure of rAAV production and vector characterization. FIG. 7C shows the packaging yield of AAV2.EGFP produced by either triple transfection (gray), AAVPure^{Mfg} 1.0 (dark blue), or AAVPure^{Mfg} 1.1 (light blue). FIG. 7D shows representative fluorescence images of HEK293 cells infected by rAAV-containing crude lysates. FIG. 7E shows AAV.EGFP capsid titers in cleared lysates determined by ELISA assay. FIG. 7F shows the full capsid ratio determined by ddPCR genome titer normalized to ELISA capsid titer. FIG. 7G shows the plasmid backbone DNA levels in rAAV products.

FIGS. 8A-8C show the application of AAVPure^{Mfg} 1.0 to different AAV serotypes. FIG. 8A depicts the schematics of plasmid components in triple transfection (left panel) and AAVPure^{Mfg} 1.0 (right panel) used to produce AAV9.EGFP and AAV8.EGFP vectors. FIG. 8B shows the comparison of AAV9.EGFP produced by either triple transfection or AAVPure^{Mfg} 1.0. FIG. 8C shows the comparison of AAV8.EGFP produced by either triple transfection or AAVPure^{Mfg} 1.0.

FIGS. 9A-9F show the asynchronous presence of pTrans and pCis plasmids causes empty capsid formation in triple transfection. FIG. 9A depicts the schematics of plasmid components in triple transfection (left panel), Trans-cis coupled triple transfection (middle panel) or AAVPure^{Mfg} 1.0 (right panel) used to produce AAV9.EGFP. FIG. 9B shows the comparison of AAV9.EGFP genome titer produced by different transfection methods. FIG. 9C shows the AAV9.EGFP capsid titer in cleared lysate determined by ELISA assay. FIG. 9D shows the full capsid ratio determined by ddPCR genome titer normalized to ELISA capsid titer. FIG. 9E shows western blotting of AAV9 VP proteins in HEK293 cells by different production methods and at indicated time points post transfection. FIG. 9F shows the dynamics of VP protein (left) and Cap (right) mRNA expression levels in HEK293 cells by different production methods and at indicated time points post transfection.

FIGS. 10A-10I depicts the development of AAVPure^{Mfg} 2.0 that utilizes standard pHelper. FIG. 10A depicts the schematics of plasmid components in AAVPure^{Mfg} 2.0. FIG. 10B shows AAV9.EGFP genome titer produced by triple transfection or AAVPure^{Mfg} 2.0 with different amount of pHelper_DBP-2A-Bxb1 spike-in. FIGS. 10C-10E show the comparison of AAV9 capsid titer (FIG. 10C), full capsid ratio (FIG. 10D), and plasmid backbone DNA levels (FIG. 10E) in cleared lysates between triple transfection and AAVPure^{Mfg} 2.0 with 1% pHelper_DBP-2A-Bxb1 spike-in. FIGS. 10F-10I show the comparison of AAV2 genome titer (FIG. 10F), capsid titer (FIG. 10G), full capsid ratio (FIG. 10H), and plasmid backbone DNA levels (FIG. 10I) in cleared lysates between triple transfection and AAVPure^{Mfg} 2.0 with 1% pHelper_DBP-2A-Bxb1 spike-in.

FIGS. 11A-11E show the development of AAVPure^{Mfg} 3.0 with a HEK293-Bxb1 cell line. FIG. 11A depicts the schematics of plasmid and cellular components in AAVPure^{Mfg} 3.0. FIG. 11B shows AAV9.EGFP genome titer produced by triple transfection or AAVPure^{Mfg} 3.0 with different monoclonal HEK293-Bxb1 cell lines. FIGS. 11C-11E show the comparison of AAV9 capsid titer (FIG. 11C), full capsid ratio (FIG. 11D), and plasmid backbone DNA levels (FIG. 11E) in cleared lysates between triple transfection and AAVPure^{Mfg} 3.0 with the monoclonal HEK293-Bxb1 cell line M19.

FIGS. 12A-12I show the application of AAVPure^{Mfg} 2.0 to suspension HEK293 cells. FIG. 12A shows a non-limiting example of the experimental procedure of AAV9.EGFP production and vector characterization. FIG. 12B shows AAV9.EGFP genome titer produced by triple transfection or AAVPure^{Mfg} 2.0 with different amount of pHelper_DBP-2A-Bxb1 spike-in. FIGS. 12C and 12D show a comparison of AAV9 capsid titer (FIG. 12C) and full capsid ratio (FIG. 12D) in cleared lysates between triple transfection and AAVPure^{Mfg} 2.0 with 1% pHelper_DBP-2A-Bxb1 spike-in. FIG. 12E shows a denaturing alkaline gel image showing the size of vector DNA purified from AAV9.EGFP in FIG. 12A. FIGS. 12F-12I shows vector DNA impurities of plasmid backbone (FIG. 12F), adenovirus helper genes (FIG. 12G), Rep and Cap genes (FIG. 12H), and host HEK293 cell genomic DNA (FIG. 12I) between triple transfection and AAVPure^{Mfg} 2.0 with 1% pHelper_DBP-2A-Bxb1 spike-in.

FIG. 13A-13C show that insertion in Rep does not impair Rep or Cap function. FIG. 13A shows a non-limiting example of the experimental procedure of western blotting and AAV2.EGFP transduction assay. FIG. 13B shows western blotting of AAV2 VPs and Rep in triple transfection with conventional pRep2/Cap2 packaging plasmid or pRep2-attR/Cap2 variants. FIG. 13C shows representative fluorescence images of HEK293 cells infected with AAV2.EGFP-containing crude lysates.

FIGS. 14A-14C show attP-ITR-Transgene-ITR-attB insertion in Rep abolishes Rep and Cap expression and function. FIG. 14A shows a schematic diagram illustrating the insertion of attP/attB-flanked Cis construct into the attR2 site in pRep2/Cap2 plasmid. FIG. 14B shows western blotting of AAV2 VPs and Rep in triple transfection or dual transfection with or without the Bxb1 gene. FIG. 14C shows the results of a Rep-dependent vector genome amplification assay.

FIGS. 15A-15B show the design and validation of pHelper-Bxb1. FIG. 15A shows a schematic of the plasmid components of the standard pHelper and modified versions carrying Bxb1. FIG. 15B shows the results of a reporter assay to test the Bxb1 recombination activity.

FIGS. 16A-16E show the development of AAVpure^{Mfg} 2.1 with pCAG-Bxb1 spike-in. FIG. 16A shows schematics of plasmid components in AAVpure^{Mfg} 2.1. FIG. 16B shows AAV9.EGFP genome titer produced by triple transfection or AAVpure^{Mfg} 2.1 with different spike-in amount of pCAG-Bxb1. FIGS. 16C-16E show the comparison of AAV9 capsid titer (FIG. 16C), full capsid ratio (FIG. 16D), and plasmid backbone DNA levels (FIG. 16E) in cleared lysates between triple transfection and AAVpure^{Mfg} 2.1 with 1% pCAG-Bxb1 spike-in.

FIGS. 17A-17E show the generation of a monoclonal HEK293-Bxb1 cell line. FIG. 17A shows a schematic diagram of Bxb1 knock-in into the AAVS1 site in HEK293 cells. FIG. 17B shows a schematic diagram, of the workflow to engineer and select HEK293-Bxb1 cells. FIG. 17C shows the non-limiting procedure of HEK293-Bxb1 monoclonal cell line generation and screening. FIG. 17D shows a schematic of a reporter assay to determine Bxb1 recombination activity. FIG. 17E shows representative fluorescent images showing naïve HEK293 cells or HEK293-Bxb1 monoclonal cell lines that were transfected with the reporter plasmids, pCMV-attP and pattB-EGFP. Images were taken 1 day post transfection.

FIG. 18 show alkaline gel images under different exposure conditions, showing AAV9.EGFP vector genome integrity.

FIG. 19 show the relative plasmid mass usage in standard triple transfection and AAVpure^{Mfg}.

### DETAILED DESCRIPTION OF INVENTION

Standard triple transfection is the most widely used method for recombinant adeno-associated virus (rAAV) production. In this method, HEK293 cells are co-transfected at roughly an equal ratio of three plasmids: the helper plasmid (pHelper) encoding adenoviral helper genes, the packaging plasmid (pTrans) encoding AAV Rep and Cap genes, and the cis-element plasmid (pCis) that carries the desired transgene flanked by inverted terminal repeats (ITRs). After triple transfection, adenoviral helper genes drive the expression of Rep and Cap genes. Rep proteins are responsible for rAAV genome replication and encapsulation, and Cap proteins—VP1, VP2 and VP3—form rAAV capsid. The ITR-flanked transgene cassette constitutes the single-stranded rAAV genome packaged within the rAAV virion. Though traditional triple transfection method has gained tremendous success in rAAV production, the huge plasmid demand, alarming backbone DNA and host genome encapsidation, and low full capsid ratio beg for substantial improvement.

rAAV produced by triple transfection typically contains 1%-10% plasmid backbone DNA (relative to transgene DNA) that is mostly derived from pCis; some reports indicate levels as high as 26.1%. These prokaryotic sequences often include highly immunogenic CpG motifs and potentially harmful open reading frames (ORFs), such as antibiotic resistance genes. It has been shown that the packaged prokaryotic DNA persists in animal tissues following rAAV administration in mice, dogs and non-human primates. These prokaryotic DNA, together with their undesired RNA and/or protein products, may trigger immune responses and cause cytostatic effects in recipients. Indeed, as little as 0.87% of plasmid backbone in rAAV was shown to cause inflammation and toxicity in non-human primates. In clinical trials, plasmid backbone DNA contaminants in rAAV was considered as a potential contributor to adverse events and led to clinical holds by FDA.

Because encapsidated prokaryotic DNA cannot be eliminated by downstream processing, mitigation strategies mainly focus on plasmid design and upstream processing. For instance, using pCis with oversized backbone that exceeds AAV packaging capacity has been shown to decrease plasmid backbone encapsidation by approximately 5-fold; including transcriptional insulators adjacent to ITRs further suppresses backbone DNA expression in transduced cells. It has been recently shown that simply reducing pCis usage in triple transfection can lower plasmid backbone encapsidation by 2- to 7-fold. However, these strategies only lead to modest improvement. Replacing plasmid with minicircle DNA or doggybone DNA that lacks plasmid backbone sequences proves to more effectively reduce prokaryotic DNA encapsidation, but their implementation was hindered by technical complexities associated with DNA manufacturing and high synthesis error rates.

In addition to prokaryotic DNA contaminants, empty capsids are also a major rAAV impurity, representing 70%-90% of total rAAV particles in crude harvest. Several studies showed that excessive empty capsids could compromise vector transduction efficiency by competitive binding with cell surface receptors. Furthermore, empty capsids can trigger or exacerbate capsid-directed immune responses, resulting in the clearance of transduced cells and further compromising therapeutic efficacy. Although advances in downstream processing have improved full capsid ratio in general, the optimal process usually needs to be individually developed for specific rAAV products, and the success highly depends on the full capsid ratio in the starting material.

Aspects of the disclosure relate to methods for minicircle dual transfection (e.g., of rAAV-encoding gene products, which significantly reduce plasmid consumption, vector impurity, and empty capsid formation, while at the same time generates comparable or higher rAAV titer). In some aspects, the disclosure relates to method for high-purity AAV vector production utilizing recombination-dependent minicircle formation and genetic coupling (AAVPure^{Mfg}). Minicircle dual transfection is also referred to as AAVPure^{Mfg}. In some embodiments, methods described herein comprise co-transfecting a cell with two circular nucleic acids: a first nucleic acid comprising a sequence encoding one or more adenovirus helper proteins and a sequence encoding a recombinase (e.g., Bxb1 recombinase) (referred to herein as ‘pHelper-recombinase’), and a second nucleic acid comprising a sequence encoding an AAV ITR-flanked transgene and a sequence encoding Rep and/or Cap proteins interspersed with recombinase recognition sites (referred to herein as ‘pTrans/Cis’). In some embodiments, the recombinase catalyzes the recombination between the recombinase recognition sites (e.g., attP and attB sites) in the pTrans/Cis, generating a nucleic acid that expresses functional AAV Rep and Cap proteins, and a minicircle DNA that comprises the only the transgene flanked by AAV ITRs (e.g., the minicircle DNA does not comprise any plasmid backbone DNA sequence).

In some embodiments, methods described herein comprise co-transfecting a cell with three circular nucleic acids: a first nucleic acid comprising a sequence encoding one or more adenovirus helper proteins and a sequence encoding a recombinase (e.g., Bxb1 recombinase) (referred to herein as ‘pHelper-recombinase’), a second nucleic acid comprising a sequence encoding an AAV ITR-flanked transgene and a sequence encoding Rep and/or Cap proteins interspersed with recombinase recognition sites (referred to herein as ‘pTrans/Cis’), and a third nucleic acid comprising one or more helper plasmids (e.g., a plasmid encoding one or more adenoviral helper factors). In some embodiments, the recombinase catalyzes the recombination between the recombinase recognition sites (e.g., attP and attB sites) in the pTrans/Cis, generating a nucleic acid that expresses functional AAV Rep and Cap proteins, and a minicircle DNA that comprises the only the transgene flanked by AAV ITRs (e.g., the minicircle DNA does not comprise any plasmid backbone DNA sequence).

In some embodiments, methods described herein comprise co-transfecting a cell with a recombinase (e.g., Bxb1) inserted into the genome of the cell (e.g., inserted into the AAVS1 safe harbor locus), with two circular nucleic acids: a first nucleic acid comprising a sequence encoding AAV ITR-flanked transgene and a sequence encoding Rep and/or Cap proteins interspersed with recombinase recognition sites (referred to herein as ‘pTrans/Cis’), and a second nucleic acid comprising one or more helper plasmids. In some embodiments, the recombinase catalyzes the recombination between the recombinase recognition sites (e.g., attP and attB sites) in the pTrans/Cis, generating a nucleic acid that expresses functional AAV Rep and Cap proteins, and a minicircle DNA that comprises the only the transgene flanked by AAV ITRs (e.g., the minicircle DNA does not comprise any plasmid backbone DNA sequence).

### Helper-Recombinase Constructs

Aspects of the disclosure relate to a circular nucleic acids comprising a first nucleic acid sequence encoding one or more adenoviral helper factors; and a second nucleic acid sequence encoding a recombinase.

As used herein, “adenoviral helper factors” refers to gene products (e.g., proteins, functional nucleic acids, etc.) that are naturally expressed by adenoviruses and that are required by dependoviruses (including AAVs) for replication and packaging of viral particles. Examples of adenovirus helper proteins include but are not limited to DNA-binding proteins, shuttle proteins, etc., including but not limited to E1A, E1B55K, E2A, E4orf6, and VA. Additional functions of adenovirus helper proteins are described, for example in Meier et al. Viruses. 2020 June; 12 (6): 662. In some embodiments, the adenoviral helper genes comprise E4, E2a and/or VA RNA transcription unit genes.

As used herein, “recombinase” refers to a protein that a family of enzymes having functional roles in homologous and site-specific recombination. In some embodiments a recombinase is a site-specific recombinase. Examples of site-specific recombinase proteins include but are not limited to Cre recombinases, Hin recombinases, Tre recombinases, and FLP recombinases, for example as described in Wang et al. Plant Cell Rep. 2011; 30 (3): 267-285. In some embodiments, the recombinase is a tyrosine recombinase. Examples of tyrosine recombinases include but are not limited to Cre, FLP, R, Lambda, HK101, and pSAM2. In some embodiments a recombinase is a serine recombinase. Examples of serine recombinase proteins include but are not limited to phiC31, Bxb1, TP901-1, and R4. In some embodiments the recombinase is Bxb1. In some embodiments, Bxb1 comprises the amino acid sequence set forth as:

(SEQ ID NO: 1)
MRALVVIRLSRVTDATTSPERQLESCQQLCAQRGWDVVGVAEDLDVSGAV
DPFDRKRRPNLARWLAFEEQPFDVIVAYRVDRLTRSIRHLQQLVHWAEDH
KKLVVSATEAHFDTTTPFAAVVIALMGTVAQMELEAIKERNRSAAHFNIR
AGKYRGSLPPWGYLPTRVDGEWRLVPDPVQRERILEVYHRVVDNHEPLHL
VAHDLNRRGVLSPKDYFAQLQGREPQGREWSATALKRSMISEAMLGYATL
NGKTVRDDDGAPLVRAEPILTREQLEALRAELVKTSRAKPAVSTPSLLLR
VLFCAVCGEPAYKFAGGGRKHPRYRCRSMGFPKHCGNGTVAMAEWDAFCE
EQVLDLLGDAERLEKVWVAGSDSAVELAEVNAELVDLTSLIGSPAYRAGS
PQREALDARIAALAARQEELEGLEARPSGWEWRETGQRFGDWWREQDTAA
KNTWLRSMNVRLTFDVRGGLTRTIDFGDLQEYEQHLRLGSVVERLHTGM
S.

In some embodiments, a nucleic acid sequence encoding a recombinase further comprises a linker sequence, for example a self-cleavable peptide-encoding sequence. In some embodiments, the self-cleaving peptide is a 2A (also known as T2A) peptide. In some embodiments, the linker sequence is an IRES sequence.

The positioning of the first nucleic acid and second nucleic acid of a circular nucleic acid may vary. In some embodiments, the first nucleic acid and second nucleic acid are positioned on the same bacterial plasmid. In some embodiments, a first nucleic acid is positioned 5′ relative to a second nucleic acid (e.g., as measured from the first nucleotide base of an open reading frame of the circular nucleic acid). In some embodiments, a first nucleic acid is positioned 3′ relative to a second nucleic acid (e.g., as measured from the first nucleotide base of an open reading frame of the circular nucleic acid). In some embodiments, the first nucleic acid sequence and second nucleic acid sequence are operably linked to the same promoter. In some embodiments, the promoter is a constitutive promoter or an inducible promoter. In some embodiments, the sequence encoding the recombinase is positioned upstream of a stop codon of the sequence encoding the one or more adenovirus helper proteins. In some embodiments a 2A-Bxb1-encoding nucleic acid sequence is located before the stop codon of an adenoviral DNA-binding protein. In some embodiments, the DNA-binding protein is an E2A helper factor. In some embodiments, 2A-Bxb1 allows for the expression of Bxb1 without the introduction of an extra promoter. In some embodiments an IRES-Bxb1-encoding nucleic acid sequence is located before the stop codon of an adenoviral DNA-binding protein. In some embodiments, the DNA-binding protein is an E2A helper factor. In some embodiments, IRES-Bxb1 allows for the expression of Bxb1 without the introduction of an extra promoter.

In some embodiments, a nucleic acid sequence encoding a recombinase further comprises a promoter. In some embodiments, the promoter is a CAG promoter.

### Trans-Cis Constructs

Aspects of the disclosure relate to a circular nucleic acids comprising a first nucleic acid sequence encoding an adeno-associated virus (AAV) Rep; a second nucleic acid sequence encoding a transgene comprising a promoter operably linked to a nucleic acid sequence encoding a gene product, flanked by AAV inverted terminal repeats (ITRs); and a third nucleic acid sequence encoding an AAV capsid protein. In some embodiments, the circular nucleic acid comprises one or more (e.g., a pair) of recombinase recognition sites positioned between the first and third nucleic acid sequences.

As used herein, a “recombinase recognition site” refers to a nucleic acid sequence that is bound by one or more recombinase proteins and undergoes strand exchange with another nucleic acid sequence due to the binding of the recombinase protein. In some embodiments, a recombinase recognition site is bound by a site-specific recombinase protein. Examples of site-specific recombinase proteins include but are not limited to Cre recombinases, Hin recombinases, Tre recombinases, and FLP recombinases, for example as described in Wang et al. Plant Cell Rep. 2011; 30 (3): 267-285. In some embodiments, the recombinase recognition site is bound by a tyrosine recombinase protein. Examples of tyrosine recombinases include but are not limited to Cre, FLP, R, Lambda, HK101, and pSAM2. In some embodiments a recombinase recognition site is bound by a serine recombinase. Examples of serine recombinase proteins include but are not limited to phiC31, Bxb1, TP901-1, and R4. In some embodiments the recombinase recognition site is a Bxb1 recognition site. Recognition sites of Bxb1 are known, and include attP, attB, attL, and attR, for example as described by Fayed et al. BMC Biotechnology 14 (1): 51 (2014), the contents of which are incorporated by reference herein. In some embodiments, a circular nucleic acid comprises two recombinase recognition sites (e.g., two Bxb1 recognition sites). In some embodiments, the recombinase recognition sites are attP and attB.

In some embodiments, a circular nucleic acid comprises a second nucleic acid sequence encodes a transgene comprising a promoter operably linked to a nucleic acid sequence encoding a gene product. In some embodiments, a transgene is flanked by AAV inverted terminal repeats (ITRs) (and may also be referred to herein as an ‘rAAV vector’). The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise, as disclosed elsewhere herein, one or more regions that encode one or more proteins and/or one or more binding sites for inhibitory nucleic acids (e.g., shRNA, miRNAs, etc.). The transgene may also comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “pTrans/Cis” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125, and variants thereof. In some embodiments, the isolated nucleic acid comprises a region (e.g., a first region) encoding an AAV2 ITR.

In some embodiments, the isolated nucleic acid further comprises one or more AAV ITRs. In some embodiments, an AAV ITR has a serotype selected from AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125, and variants thereof. In some embodiments, an AAV ITR is a mutant ITR (mTR) that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ΔTRS ITR). Without wishing to be bound by any particular theory, a rAAV vector comprising an ITR lacking a functional TRS produces a self-complementary rAAV vector, for example as described by McCarthy (2008) Molecular Therapy 16 (10): 1648-1656.

In some embodiments, a second nucleic acid sequence is positioned 3′ with respect to a first nucleic acid sequence (e.g., a nucleic acid sequence encoding an adeno-associated virus (AAV) Rep protein). In some embodiments, a second nucleic acid is positioned within an intron of a first nucleic acid (e.g., an intron of a nucleic acid sequence encoding an AAV Rep protein). In some embodiments, the second nucleic acid sequence is flanked by recombinase recognition sites (e.g., attP and attB recombinase recognition sites).

In some embodiments, a circular nucleic acid comprises a third nucleic acid sequence encoding an AAV capsid protein (e.g., an AAV Cap gene). In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments the ratio of VP1:VP2:VP3 is about 1:1:10. In some embodiments, the functions of the capsid proteins are to protect the viral genome, deliver the genome and interact with the host. In some aspects, capsid proteins deliver the viral genome to a host in a tissue specific manner. The capsid protein may be any AAV capsid protein. Examples of AAV capsid proteins include but are not limited to AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125. In some embodiments, the capsid protein is an AAV2 capsid protein. In some embodiments, the capsid protein is an AAV5 capsid protein. In some embodiments, the capsid protein is an AAV9 capsid protein.

In some embodiments, Bxb1 recombinase catalyzes recombination between attP and attB sites in a circular nucleic acid encoding a transgene and Rep and/or Cap proteins, generating a functional protein that comprises an attR and expresses functional Rep and Cap (e.g., AAV capsid) proteins (pTrans-attR), and a minicircle that comprises an attL recognition site positioned between an ITR-flanked transgene without any plasmid backbone sequences (minicircle pCis). In some embodiments, the circular nucleic acid comprising the attR further comprises a promoter. In some embodiments, the promoter is a P40 promoter. In some embodiments, the attR is positioned between the P40 promoter and the end of an intron (e.g., the intron of the first nucleic acid sequence). In some embodiments, a first nucleic acid sequence and second nucleic acid sequence are in a first open reading frame, and a third nucleic acid sequence is in a second open reading frame. In some embodiments, after recombination by a Bxb1 protein, a first nucleic acid sequence (e.g., a nucleic acid sequence encoding an AAV Rep protein) and a third nucleic acid sequence (e.g., a nucleic acid sequence encoding an AAV capsid protein) are positioned within the same reading frame. In some embodiments, the first nucleic acid sequence and the second nucleic acid sequence are operably linked to the same promoter.

### Helper Plasmid

As used herein the term “adenoviral helper factors” also known as “helper plasmids” or “pHelper” refers to adenoviral helper genes, gene products (e.g., proteins, functional nucleic acids, etc.) that are naturally expressed by adenoviruses and that are required by dependoviruses (including AAVs) for replication and packaging of viral particles. Examples of adenovirus helper proteins include but are not limited to DNA-binding proteins, shuttle proteins, etc., including but not limited to E1A, E1B55K, E2A, E4orf6, and VA. Additional functions of adenovirus helper proteins are described, for example in Meier et al. Viruses. 2020 June; 12 (6): 662. In some embodiments, the adenoviral helper genes comprise E4, E2a and/or VA RNA transcription unit genes. In some embodiments, the pHelper is AdDeltaF6.

### Nucleic Acids

As used herein, the term “nucleic acid” refers to polymers of linked nucleotides, such as DNA, RNA, etc. In some embodiments, proteins and nucleic acids of the disclosure are isolated. In some embodiments, the DNA of a transgene is transcribed into a messenger RNA (mRNA) transcript. As used herein, the term “isolated” means artificially produced (e.g., an artificially produced nucleic acid, or an artificially produced protein, such as a capsid protein). As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been artificially produced (e.g., by chemical synthesis, by recombinant DNA technology, etc.) As used herein, a “transgene” is a nucleic acid sequence, which is not homologous to vector sequences, which encodes a polypeptide, protein, functional RNA molecule (e.g., miRNA, miRNA inhibitor) or other gene product, of interest. In some embodiments, a transgene encodes a therapeutic protein or therapeutic functional RNA. Examples of therapeutic proteins include toxins, enzymes (e.g., kinases, phosphorylases, proteases, acetylases, deacetylases, methylases, demethylases, etc.) growth factors, interleukins, interferons, anti-apoptosis factors, cytokines, anti-diabetic factors, anti-apoptosis agents, coagulation factors, anti-tumor factors, and anti-proliferative proteins. The nucleic acid coding sequence is operatively linked to regulatory components in a manner which permits transgene transcription, translation, and/or expression in a cell of a target tissue.

Thus, the disclosure embraces the delivery of vectors encoding one or more peptides, polypeptides, or proteins, which are useful for the treatment or prevention of disease states in a mammalian subject. Exemplary therapeutic proteins include one or more polypeptides selected from the group consisting of growth factors, interleukins, interferons, anti-apoptosis factors, cytokines, anti-diabetic factors, anti-apoptosis agents, coagulation factors, anti-tumor factors, enzymes, and anti-proliferative proteins. Other non-limiting examples of therapeutic proteins include BDNF, CNTF, CSF, EGF, FGF, G-SCF, GM-CSF, gonadotropin, IFN, IFG-1, M-CSF, NGF, PDGF, PEDF, TGF, VEGF, TGF-B2, TNF, prolactin, somatotropin, XIAP1, IL-1, IL-2, IL-3, IL-4, IL-5, IL-6, IL-7, IL-8, IL-9, IL-10, IL-10 (187A), viral IL-10, IL-11, IL-12, IL-13, IL-14, IL-15, IL-16 IL-17, and IL-18.

The nucleic acids disclosed herein may comprise a transgene to be transferred to a subject to treat a disease associated with reduced expression, lack of expression or dysfunction of the native gene. Exemplary genes and associated disease states include, but are not limited to: glucose-6-phosphatase, associated with glycogen storage deficiency type 1A; phosphoenolpyruvate-carboxykinase, associated with Pepck deficiency; galactose-1 phosphate uridyl transferase, associated with galactosemia; phenylalanine hydroxylase, associated with phenylketonuria; branched chain alpha-ketoacid dehydrogenase, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; omithine transcarbamylase, associated with omithine transcarbamylase deficiency; argininosuccinic acid synthetase, associated with citrullinemia; low density lipoprotein receptor protein, associated with familial hypercholesterolemia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotinidase, associated with biotinidase deficiency; beta-glucocerebrosidase, associated with Gaucher disease; beta-glucuronidase, associated with Sly syndrome; peroxisome membrane protein 70 kDa, associated with Zellweger syndrome; porphobilinogen deaminase, associated with acute intermittent porphyria; alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin for the treatment of diabetes.

The following are further non-limiting examples of proteins that may be encoded by transgenes disclosed herein to treat a disease associated with reduced expression, lack of expression or dysfunction of the native gene: a-galactosidase, acid-glucosidase, adiopokines, adiponectin, alglucosidase alfa, anti-thrombin, ApoAV, ApoCII, apolipoprotein A-I (APOA1), arylsulfatase A, arylsulfatase B, ATP-binding cassette transporter A1 (ABCA1), ABCD1, CCR5 receptor, erythropoietin, Factor VIII, Factor VII, Factor IX, Factor V, fetal hemoglobin, beta-globin, GPI-anchored HDL-binding protein (GPI-HBP) I, growth hormone, hepatocyte growth factor, imiglucerase, lecithin-cholesterol acyltransferase (LCAT), leptin, LDL receptor, lipase maturation factor (LMF) 1, lipoprotein lipase, lysozyme, nicotinamide dinucleotide phosphate (NADPH) oxidase, Rab escort protein-1 (REP-1), retinal degeneration slow (RDS), retinal pigment epithelium-specific 65 (RPE65), rhodopsin, T cell receptor alpha or beta chains, thrombopoeitin, tyrosine hydroxylase, VEGF, von heldebrant factor, von willebrand factor, and X-linked inhibitor of apoptosis (XIAP).

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically active polypeptide product (e.g., a therapeutic protein or therapeutic minigene) or inhibitory RNA (e.g., shRNA, miRNA, amiRNA, miRNA inhibitor) from a transcribed gene.

### Viral Vectors

Viral vectors present a powerful tool for the delivery of plasmids and genetic material into cells. Adapting plasmid DNA for use with virus-mediated delivery has provided numerous advantages for research, including the delivery of genetic information in traditionally hard-to-transfect cells, such as neurons. Viruses naturally infect host cells and direct them to reproduce the viral genome. Scientists have taken advantage of this process by providing the virus with alternate genomes (e.g., plasmids encoding a nucleic acid or transgene), which can then be replicated once the virus has infected a host cell. In short, researchers can introduce plasmids into a host cell to generate recombinant virus.

For safety reasons, viral genomes used in research and drug development have been modified through the removal of certain genes that are required for viral replication. These genes are usually divided among numerous “accessory plasmids” which must also be present in the cell for a viral particle to be produced. The production of viral particles comprising nucleic acid(s) of interest, along with the viral genome, by a host cell is herein referred to as “packaging”. The process for the delivery and packaging of nucleic acids into viral genomes varies depending on the viral genome the nucleic acid is encoded in and will be discussed in greater detail for each viral vector below.

Recombinant adeno-associated virus (rAAV) particles are produced by introducing into a host cell, a cis-element nucleic acid comprising a transgene, a helper nucleic acid encoding adenoviral helper genes, and a packaging nucleic acid encoding Rep and/or Cap genes. A cis-element nucleic acid comprising a transgene may comprise a transgene flanked by adeno-associated virus (AAV) inverted terminal repeats (ITRs). In some embodiments, a helper nucleic acid encoding adenoviral helper genes comprises genes that mediate AAV replication (e.g., AAV E4, E2a and/or VA genes). In some embodiments, a packaging nucleic acid encodes one or more Rep genes. In some embodiments, a packaging nucleic acid encodes one or more Cap genes.

In some embodiments, rAAV particles are produced using methods described by the disclosure, e.g., by introducing into a host cell two circular nucleic acids as described herein. As used herein, the term “recombinant virus” or “recombinant viral particle” refers to a particle produced in a host cell which encapsulates nucleic acid produced from exogenous DNA inserted into the host cell genome is, has been introduced.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells. The skilled artisan will appreciate that in methods described by the disclosure, a host cell may be transfected with 2, 3, 4, 5, 6, 7, 8, 9, 10, or more isolated nucleic acids.

In some aspects, the disclosure provides minicircle dual transfected host cells. In some embodiments “minicircle DNA” or a “minicircle” is a small circular replicon. In some embodiments, a minicircle does not comprise any bacteria-derived DNA. As used herein, “bacteria-derived DNA” refers to DNA sequences that are obtained from or identical to DNA sequences present in a bacterial plasmid. In some embodiments, a minicircle does not comprise any bacteria-derived DNA from an AAV production plasmid, for example “pAAV” plasmids, as described by AddGene (www.addgene.org/viral-vectors/aav/). In some aspects, the disclosure provides a circular nucleic acid (e.g., a minicircle) comprising a transgene comprising a promoter operably linked to a nucleic acid sequence encoding a gene product, flanked by AAV inverted terminal repeats (ITRs), and an attL recombinase recognition site positioned between the AAV ITRs.

“Recombinant AAV (rAAV) vectors” are typically composed of, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The components to be cultured in the host cell to package a rAAV vector in an AAV capsid may be provided to the host cell in trans. In some embodiments, the components to be cultured in the host cell to package a rAAV vector in an AAV capsid comprise: (i) a nucleic acid comprising a sequence encoding one or more adenovirus helper proteins and a sequence encoding a recombinase (pHelper-recombinase); and (ii) a nucleic acid comprising a transgene and a sequence encoding Rep and/or Cap proteins (pTrans/Cis). Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

### Cell

A “host cell” refers to any cell that harbors, or is capable of harboring, a substance of interest or of packaging the nucleic acid of interest into a viral particle. Often a host cell is a mammalian cell. Examples of host cells include human cells, mouse cells, rat cells, dog cells, cat cells, hamster cells, monkey cells, insect cells, plant cells, or bacterial cells. Examples of insect cells include but are not limited to Spodoptera frugiperda (e.g., Sf9, Sf21), Spodoptera exigua, Heliothis virescens, Helicoverpa zea, Heliothis subflexa, Anticarsia gemmatalis, Trichopulsia ni (e.g., High-Five cells), Drosophila melanogaster (e.g., S2, S3), Antheraea eucalypti, Bombyx mori, Aedes alpopictus, Aedes aegyptii, and others. Examples of bacterial cells include, but are not limited to Escherichia coli, Corynebacterium glutamicum, and Pseudomonas fluorescens. Examples of yeast cells include but are not limited to Saccharomyces cerevisiae, Saccharomyces pombe, Pichia pastoris, Bacillus sp., Aspergillus sp., Trichoderma sp., and Myceliophthora thermophila C1. Examples of plant cells include but are not limited to Nicotiana sp., Arabidopsis thaliana, Mays zea, Solanum sp., or Lemna sp.

In some embodiments, a host cell is a mammalian cell. Examples of mammalian cells include Henrietta Lacks tumor (HeLa) cells and baby hamster kidney (BHK-21) cells. In some embodiments, a host cell is a human cell, for example a HEK293T cell. A host cell may be used as a recipient of one or more viral transfer vectors and one or more accessory plasmids. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

In some embodiments, the host cell is transfected with a Bxb1-expressing plasmid. In some embodiments, the host cell comprises a Bxb1-expressing plasmid. In some embodiments the host cell is a HEK293 cell. In some embodiments, the host cell that comprises the Bxb1-expressing plasmid is a HEK293-Bxb1 cell. In some embodiments, the Bxb1-expressing plasmid is inserted into the host cell genome using any acceptable method in the art. In some embodiments, the Bxb1-expressing plasmid is inserted into the host cell genome using CRISPR-mediated homology directed repair (HDR) strategy. In some embodiments, using the CRISPR-HDR strategy the Bxb1-expressing plasmid is inserted into the AAVS1 safe harbor locus.

In some embodiments, Bxb1 is integrated into the genome of a cell. In some embodiments, the Bxb1-expressing plasmid is inserted into the host cell genome using any acceptable method in the art. In some embodiments, the Bxb1-expressing plasmid is inserted into the host cell genome using CRISPR-mediated homology directed repair (HDR) strategy. In some embodiments, the cell is a host cell. In some embodiments, the cell is a HEK293 cell. In some embodiments, Bxb1 is integrated into the AAVS1 safe harbor locus of the cell.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

In some embodiments, the cell line is a HEK293 cell line that stably expresses Bxb1.

As used herein, the terms “recombinant cell” refers to a cell into which an exogenous DNA segment, such as DNA segment that leads to the transcription of a biologically active polypeptide or production of a biologically active nucleic acid such as an RNA, has been introduced.

In some embodiments, a host cell comprises a composition comprising: (i) a nucleic acid comprising a sequence encoding one or more adenovirus helper proteins and a sequence encoding a recombinase; and (ii) a nucleic acid comprising a transgene and a sequence encoding Rep and/or Cap proteins.

Method for Producing Recombinant Adeno-Associated Virus (rAAV)

In some aspects, the present disclosure provides a method for manufacturing rAAV, the method comprising introducing into a host cell an rAAV production system comprising a first circular nucleic acid comprising a first nucleic acid sequence encoding one or more adenoviral helper factors; and a second nucleic acid sequence encoding a recombinase; and a second circular nucleic acid comprising a first nucleic acid sequence encoding an adeno-associated virus (AAV) Rep; a second nucleic acid sequence encoding a transgene comprising a promoter operably linked to a nucleic acid sequence encoding a gene product, flanked by AAV inverted terminal repeats (ITRs); and a third nucleic acid sequence encoding an AAV capsid protein.

In some embodiments, the rAAV production method described herein produces a comparable or higher titer of rAAV with reduced plasma demand relative to other methods (e.g., other known rAAV production methods in the art, for example the triple transfection method). In some embodiments, the titer of rAAV is increased by 0.1%, 0.2%, 0.3%, 0.4%, 0.5%, 1%, 2%, 3%, 4%, 5%, 6%, 7%, 8%, 9%, 10%, 20%, 30%, 40%, 50%, 60%, 70%, 80%, 90%, 100%, 150%, 200%, 250%, 300%, 350%, 400%, 450%, or 500% relative to other methods (e.g., other known rAAV production methods in the art, for example the triple transfection method). In some embodiments, the titer of rAAV is increased by 0.1-1%, 1%-10%, 10%-50%, 50%-100%, 100%-150%, 150%-200%, 200%-250%, 250%-300%, 300%-350%, 350%-400%, 400%-450%, or 450%-500% relative to other methods (e.g., other known rAAV production methods in the art, for example the triple transfection method).

In some embodiments, the rAAV production method described herein reduces the amount of plasmid backbone encapsidation relative to other methods (e.g., other known rAAV production methods in the art, for example the triple transfection method) by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In some embodiments, the rAAV production method described herein reduces the amount empty capsid formation relative to other methods (e.g., other known rAAV production methods in the art, for example the triple transfection method) by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

The recombinant AAV vector, rep sequences, cap sequences, recombinase, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

### EXAMPLES

### Example 1: Comparison Between Standard Triple Transfection and Minicircle Dual Transfection

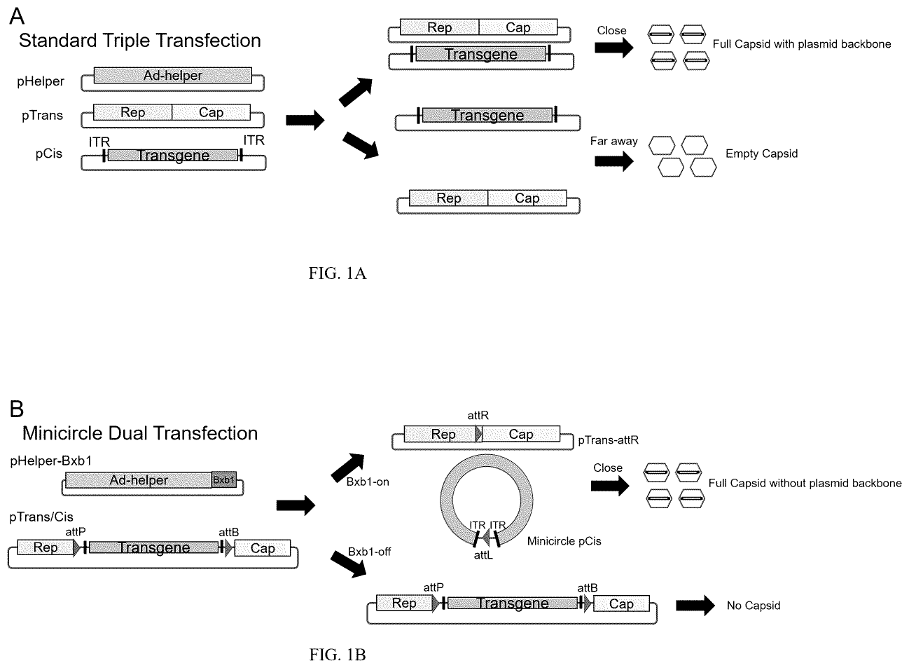

In standard triple transfection (FIG. 1A), roughly equal moles of Ad-helper plasmid (pHelper), packaging plasmid (pTrans), and cis-element plasmid encoding the ITR-flanked transgene (pCis) is co-transfected into HEK293 cells to generate desired rAAV. During this process, a substantial amount of ITR-containing plasmid backbone DNA is encapsidated into the rAAV particle. However, ITR sequences are the only required DNA sequence for rAAV packaging. In addition, due to the inherent limitations of plasmid transfection, the distribution of the three rAAV production plasmids is not even within each cell. There are many cells containing no pCis plasmid, or the pCis plasmid may be physically far away from the pTrans plasmid in the nucleus, leading to the generation of empty capsids (FIG. 1A).

This example describes a minicircle dual transfection strategy that overcomes the limitations mentioned above (FIG. 1B). These methods comprise a system of two plasmids: ‘pHelper-recombinase’ (e.g., pHelper-Bxb1), which provides adenoviral helper genes and a recombinase (e.g., Bxb1) in one plasmid, and ‘pTrans/Cis’, which provides Trans and Cis elements (e.g., Rep, Cap, and transgene-encoding sequences) separated by recombinase recognition sites (e.g., attB, attP) in another plasmid. When pHelper-Bxb1 and pTrans/Cis are co-transfected into HEK293 cells, Bxb1 catalyzes the recombination between attP and attB sites in pTrans/Cis, generating a new functional pTrans-attR plasmid that expresses Rep and Cap proteins, and a minicircle DNA, referred to herein as ‘pCis’, which contains only ITR-flanked transgene without any plasmid backbone sequences (FIG. 1B). In this way, the packaged full AAV capsids are devoid of plasmid backbone DNA encapsidation. In the absence of recombinase expression (e.g., if HEK293 cells are transfected with only pTrans/Cis), the ITR-flanked transgene embedded in the Rep gene of the pTrans/Cis plasmid serves as a disrupting insertion that inhibits Cap expression (e.g., production of capsid proteins), thus avoiding empty capsid formation (FIG. 1B).

### Example 2: Identification of attR Knock-In Site

In minicircle dual transfection, a major technical challenge is the knock-in of attP-ITR-transgene-ITR-attB cassette in pTrans (FIG. 1B). This cassette inhibits Cap expression before Bxb1-mediated recombination. After Bxb1-mediated recombination, the pTrans-attR allows for functional Rep expression with in-frame attR and restores Cap expression; both Rep and Cap proteins are required for rAAV production.

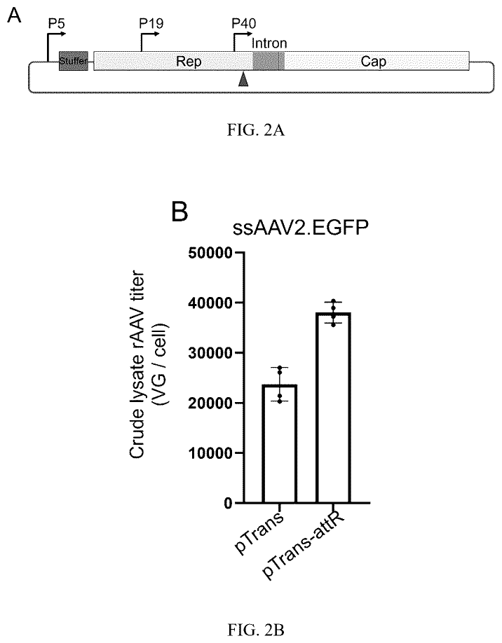

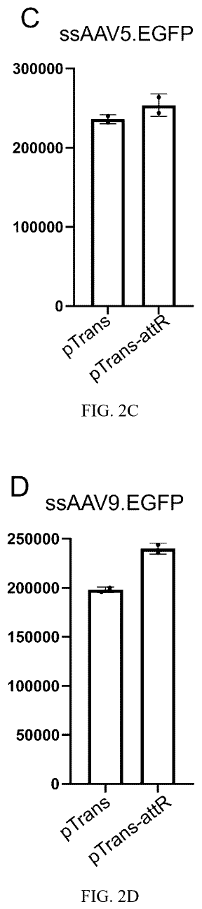

A cassette was inserted between P40 and intron (FIG. 2A). This is because Cap expression is driven by the P40 promoter, and it was surprisingly observed that the presence of this intron enables a 1:1:10 ratio of VP1:VP2:VP3 to be expressed. The P5 and p19 promoters drive expression of Rep protein isoforms. After screening multiple sites in the region between P40 and intron of pTrans, one site was found that is amenable to attR knock-in (i.e., attR knock-in does not compromise Rep function). Surprisingly, attR knock-in mildly improves rAAV production for multiple serotypes, AAV2 (FIG. 2B), AAV5 (FIG. 2C), and AAV9 (FIG. 2D). Concerning pHelper-Bxb1, a self-cleavable 2A peptide fused to the Bxb1 recombinase encoding sequence (‘2A-Bxb1’) was inserted before the stop codon of adenoviral DNA-binding protein (DBP), a critical helper gene in pHelper. In this way, Bxb1 is expressed without introducing an extra promoter to the expression construct.

### Example: 3: Minicircle Dual Transfection Produces Comparable or Higher Titer of rAAV with Reduced Plasmid Demand

In standard triple transfection, an equal mass of three plasmids (pHelper, pTrans and pCis) are co-transfected to HEK293 cells to produce rAAV. In minicircle dual transfection, the molar amount of pHelper-Bxb1 and pTrans/Cis is the same as pHelper and pTrans used in standard triple transfection, respectively. rAAV was harvested from HEK293 cell crude lysate in 12-well plate production scale. DNAse-resistant rAAV titer (GC/cell) was determined by ddPCR.

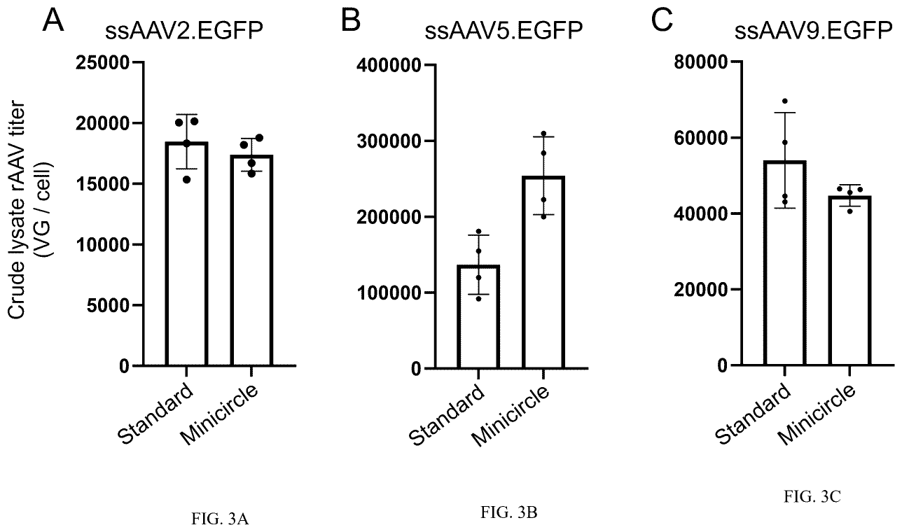

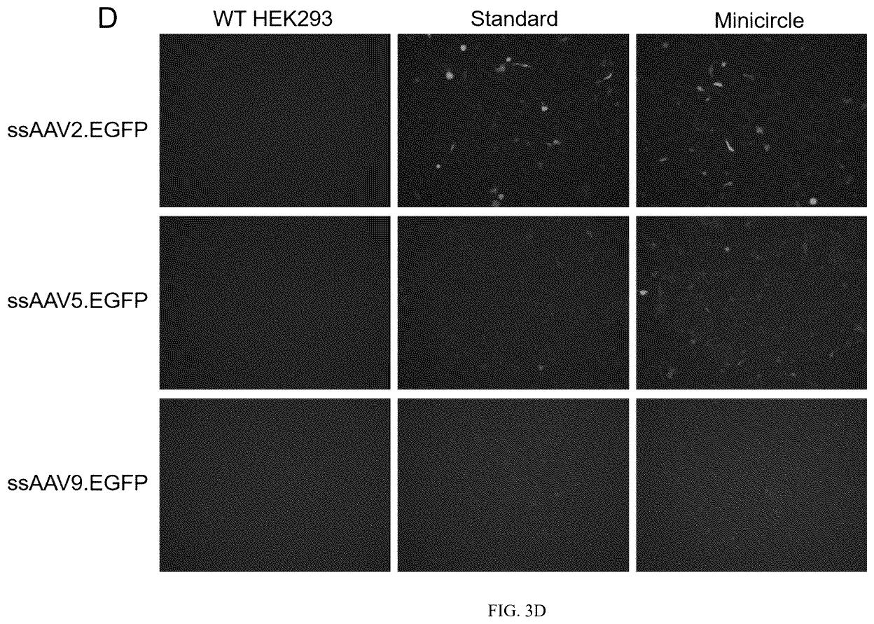

Minicircle dual transfection generates comparable or higher rAAV yield in different serotypes while saving roughly ½ plasmids by mole or ⅕ plasmids by mass (Table 1). Compared with standard triple transfection, rAAV titer was similar when packaging single-stranded (ss) AAV2.EGFP (FIG. 3A) or ssAAV9.EGFP (FIG. 3C). Interestingly, rAAV titer was increased by around 2 folds when packaging ssAAV5.EGFP (FIG. 3B). In addition, these rAAV vectors show similar potency as those produced by standard triple transfection in HEK293 cells (FIG. 3D).

TABLE 1
Plasmid usage in standard triple transfection and minicircle dual transfection

| | | | | Minicircle Dual Transfection | |
|---|---|---|---|---|---|
| | Standard Triple Transfection | | | pHelper- | |
| Scale | pHelper | pTrans | pCis | Bxb1 | pTrans/Cis |
| 12-Well | 0.5 μg | 0.5 μg | 0.5 μg | 0.55 μg | 0.65 μg |
| Plate | (52.5 fmol) | (101 fmol) | (157.6 fmol) | (52.5 fmol) | (101 fmol) |
| 10 Roller | 1.5 mg | 1.5 mg | 1.5 mg | 1.66 mg | 1.96 mg |
| Bottle | (157.4 pmol) | (303 pmol) | (472.8 pmol) | (157.4 pmol) | (303 pmol) |

### Example 4: Minicircle Dual Transfection Generates rAAV with Dramatically Reduced Plasmid Backbone Encapsidation

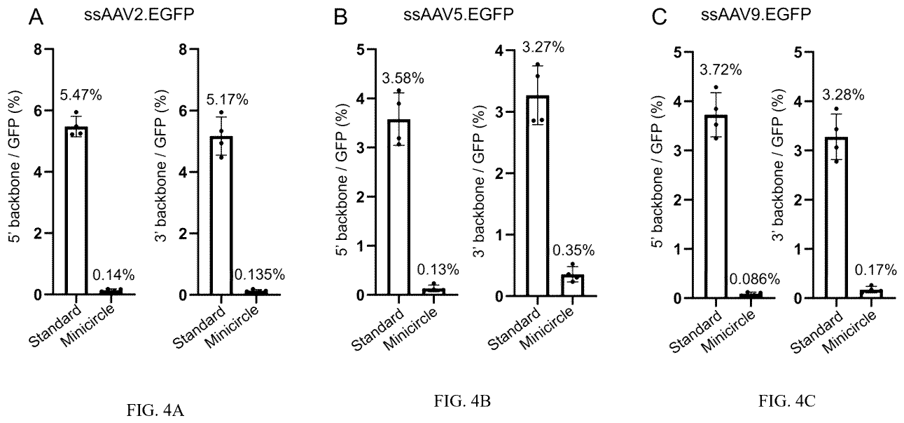

Minicircle dual transfection dramatically reduces plasmid backbone DNA encapsidation. A duplex digital droplet PCR (ddPCR) was used to assay backbone ratio with one probe targeting EGFP transgene, the other targeting either 5′ or 3′ plasmid backbone. The results show that the ratio of encapsidated plasmid backbone DNA was reduced by ˜40 folds in AAV2 (FIG. 4A), 9-28 folds in AAV5 (FIG. 4B) and 19-43 folds in AAV9 (FIG. 4C).

### Example 5: Minicircle Dual Transfection Generates rAAV with Reduced Empty Capsid Formation

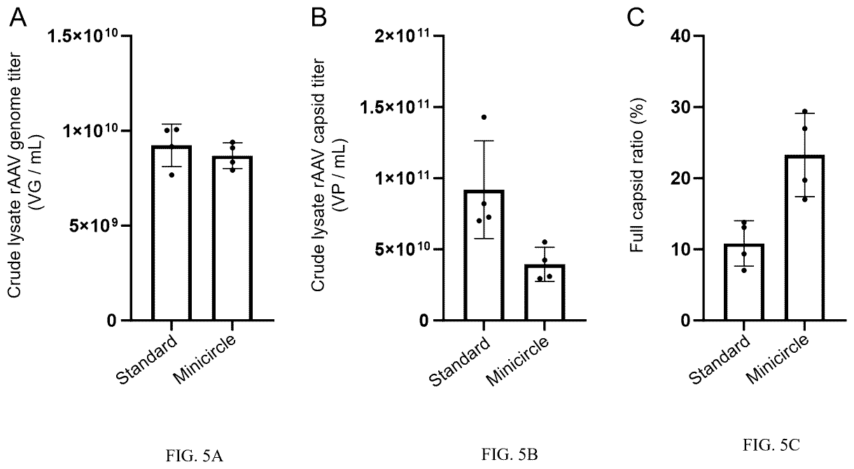

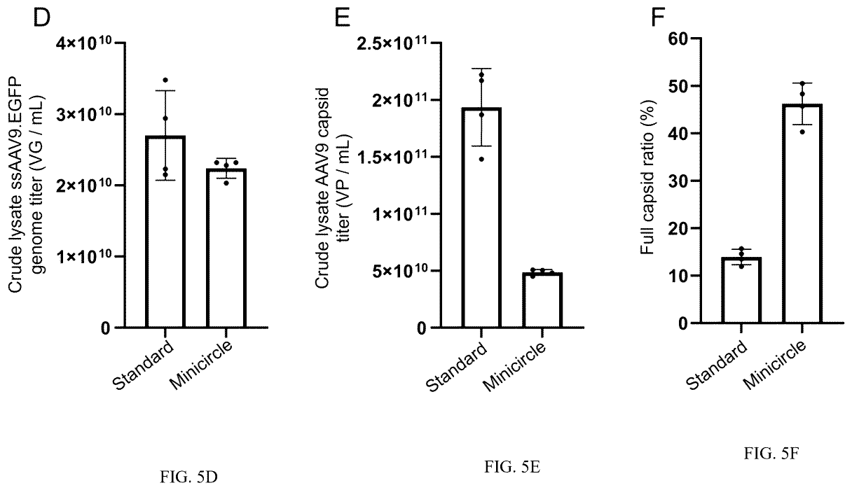

Importantly, empty capsid formation was significantly mitigated by 2-4-fold in minicircle dual transfection. When packaging ssAAV2.EGFP or ssAAV9.EGFP, the rAAV titer was comparable between standard triple transfection and minicircle dual transfection (FIGS. 5A, 5D). By contrast, the capsid titer was reduced by 2.3 folds in AAV2 and 4 folds in AAV9 when using minicircle dual transfection (FIGS. 5B, 5E). Consequently, the full capsid ratio was more than doubled from 10.8% to 23.3% in AAV2, more than tripled from 13.9% to 46.2% in AAV9 (FIGS. 5C, 5F).

In summary, minicircle dual transfection effectively packages high-quality rAAV by saving around 20% of total plasmid cost. The AAV vector products show superior quality with up to 43 folds reductions in plasmid backbone DNA contamination and more than 2-fold increase in full capsid formation. The methods herein are broadly applicable to packaging different capsid serotypes.

### Example 6: Design and Characterization of AAVPure^{Mfg} Components

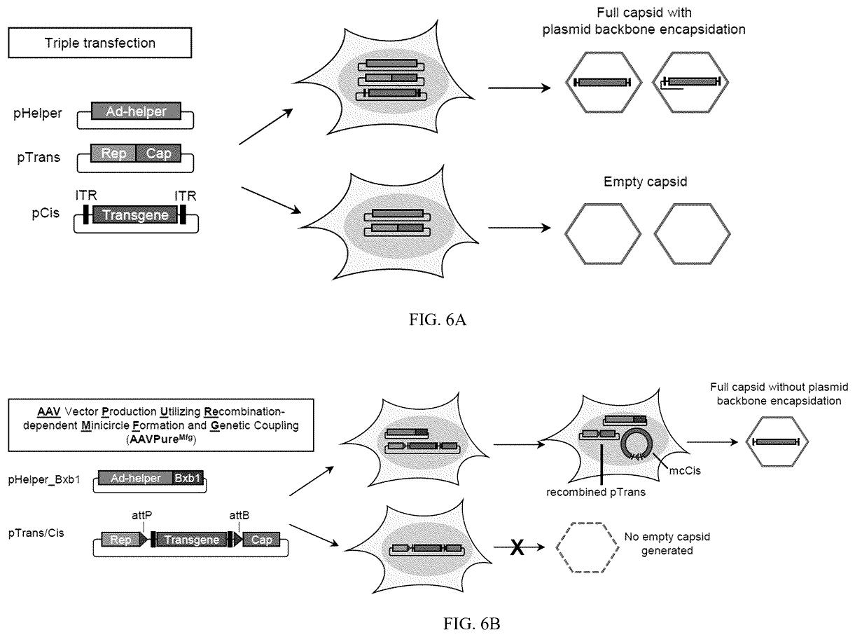

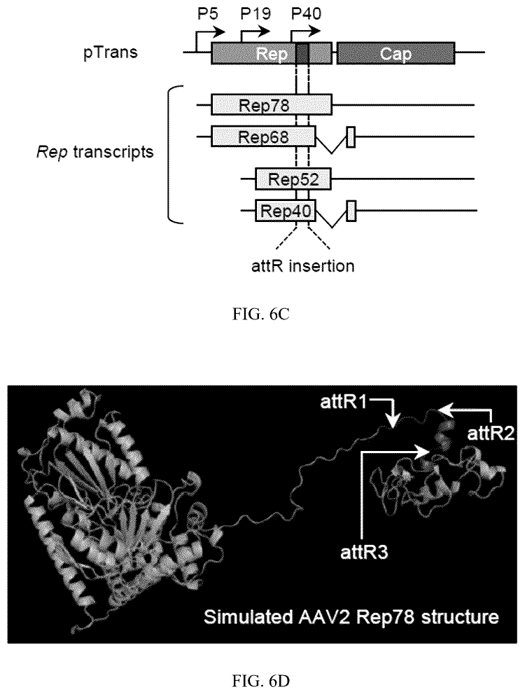

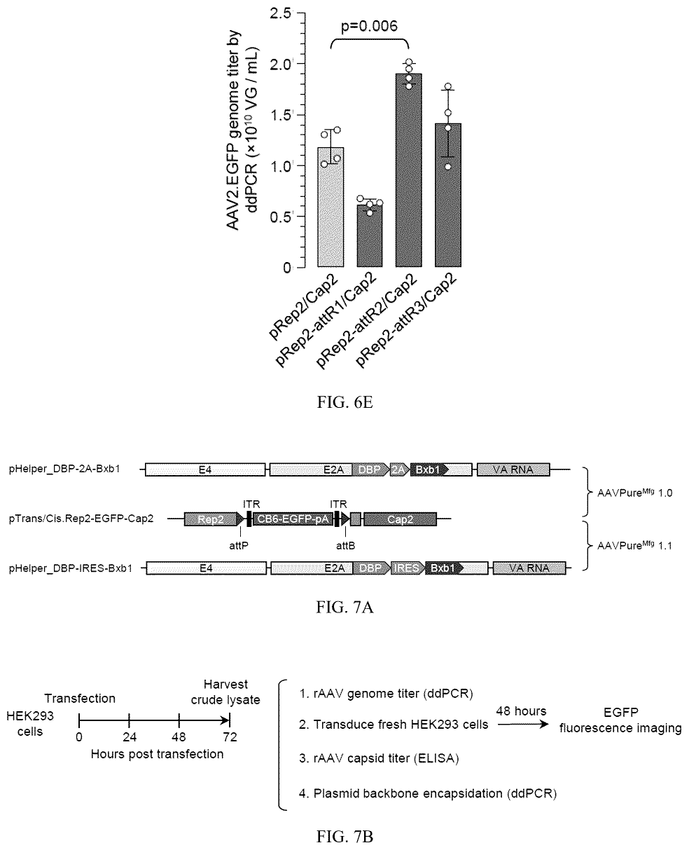

As disclosed herein, empty capsids can be generated due to the asynchronous presence of pTrans and pCis during triple transfection in HEK293 cells. Specifically, functional expression of Rep and Cap from pTrans in the absence of pCis is a major source of empty capsid formation (FIG. 6A). Therefore, AAVPure^{Mfg} was designed aiming to ensure the co-existence of the trans and cis constructs in the same nucleus following transfection. It comprises two plasmids (FIG. 6B): pHelper-Bxb1 that delivers adenoviral helper genes and the recombinase Bxb1, and pTrans/Cis with an attP/attB-flanked cis construct inserted into the 3′ region of Rep gene in the standard pTrans²⁸. Upon co-transfection into HEK293 cells, Bxb1-mediated attP/attB recombination reconstitutes pTrans and generates a minicircle Cis construct (mcCis) devoid of prokaryotic DNA sequence; the recombined pTrans affords Rep and Cap expression, and the mcCis contains ITR-flanked transgene, serving as the replication template to generate high-purity vector genomes without plasmid backbone DNA. Importantly, when HEK293 cells receive only pTrans/Cis, the ITR-flanked transgene cassette embedded in the Rep gene serves as a disrupting insertion that abolishes Cap expression, thus preventing empty capsid formation (FIG. 6B). The success of AAVPure^{Mfg} hinges on two key gene expression control events: (1) when Bxb1-mediated recombination occurs, the reconstituted pTrans should enable functional Rep and Cap expression for vector packaging, and (2) in the absence of Bxb1 (i.e., no recombination), the inserted cis construct must block Cap expression to prevent empty capsid formation.

To achieve the first control event, one challenge is that attP/attB recombination leaves an attR sequence within the Rep open reading frame (ORF), potentially compromising Rep and/or Cap expression. Therefore, an in-frame attR site was inserted between the P40 promoter (driving Cap transcription) and the intron (FIG. 6C). This insertion region was strategically chosen for two reasons: (1) following attP/attB recombination, although the resulting attR sequence is transcribed and included in Cap transcripts, it does not disrupt intron integrity and maintains normal Cap mRNA splicing that is essential to generate capsid protein isoforms (i.e., VP1, VP2, VP3), and (2) this insertion region in the Rep protein is predicated to be disordered (FIG. 6D) and has been shown to be amenable to mutagenesis, and therefore may tolerate the additional residues encoded by attR.

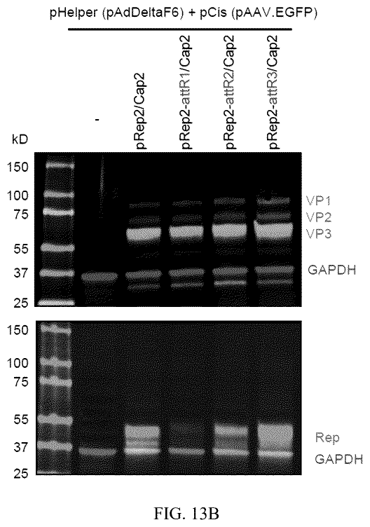

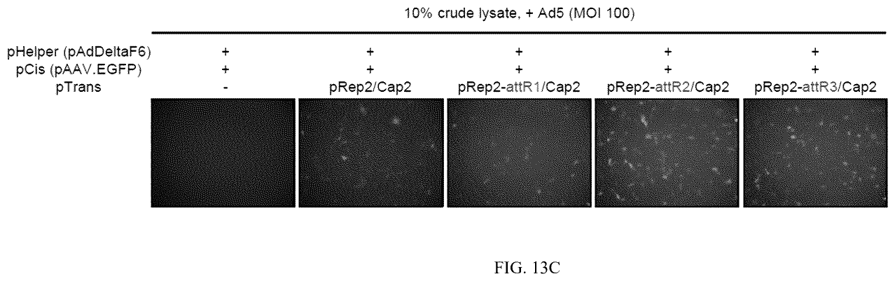

The pTrans was modified for packaging AAV2 serotype vector (pRep2/Cap2) by inserting attR at three positions individually within the chosen region (pRep2-attR1/Cap2. pRep2-attR2/Cap2, pRep2-attR3/Cap2). As expected, all three pRep2-attR/Cap2 variants afforded rAAV production following a standard triple transfection scheme (FIGS. 6D and 6E), without altering the VP isoforms ratio or abundance as determined by western blot analysis (FIGS. 13A and 13B). Consistent with in-frame attR insertion. Rep-attR fusion proteins showed a slight molecular weight shift compared with the wildtype Rep expressed from unmodified pTrans (FIG. 13B, bottom panel). When equal volume of rAAV-containing cell lysates was used in an in vitro transduction assay (FIG. 13A), reporter gene expression well correlated with rAAV titers (compare FIG. 13C with FIG. 6E), consistently showing that pRep2-attR2/Cap2 slightly outperformed the other two variants. Therefore, the attR2 insertion position was focused on in the following studies.

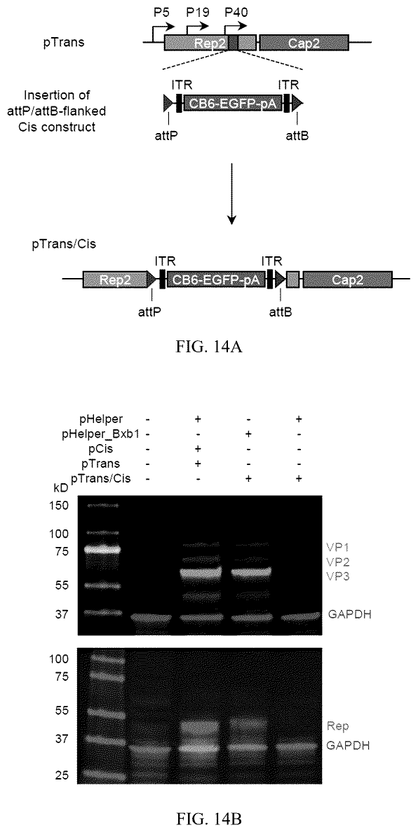

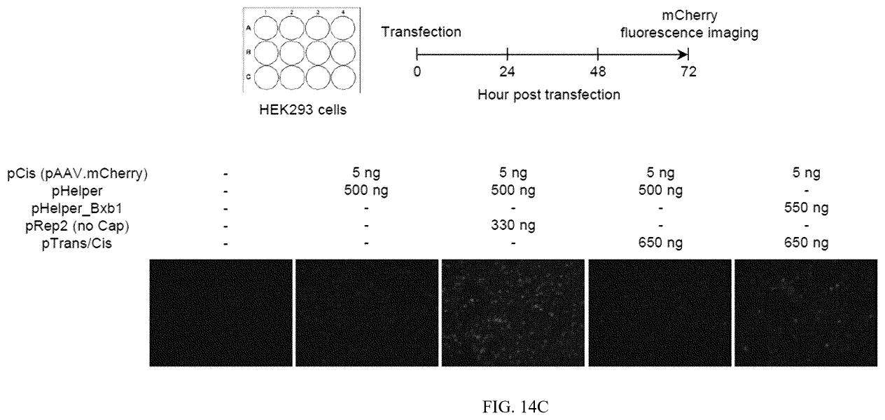

To achieve the second control event, affR2 was replaced with the attP/attB-flanked cis construct (i.e., attP-ITR-CB6-EGFP-pA-ITR-attB) in the same orientation as Rep (FIG. 14A), so that the polyadenylation signal (pA) within the transgene cassette could prematurely terminate Cap transcription and blunt VP expression (FIG. 14B, top panel, lane 5). It was observed that Rep protein expression from pTrans/Cis was also abolished (FIG. 14B, bottom panel, lane 5). To investigate whether pTrans/Cis could express residual amount of functional Rep variants that were not detectable by western blot, a Rep-dependent vector genome amplification assay was devised (FIG. 14C). In this assay, a small amount of pCis (p.AAV.mCherry) was co-transfected with pHelper and pRep (no Cap), so that the vector genome (i.e., ITR-flanked mCherry cassette) was replicated, resulting in increased mCherry expression (FIG. 14C, compare panel 2 and panel 3). In contrast, replacing pRep (no Cap) with pTrans/Cis failed to amplify mCherry fluorescence signal (FIG. 14C, panel 4), demonstrating successful silencing of Rep in pTrans/Cis.

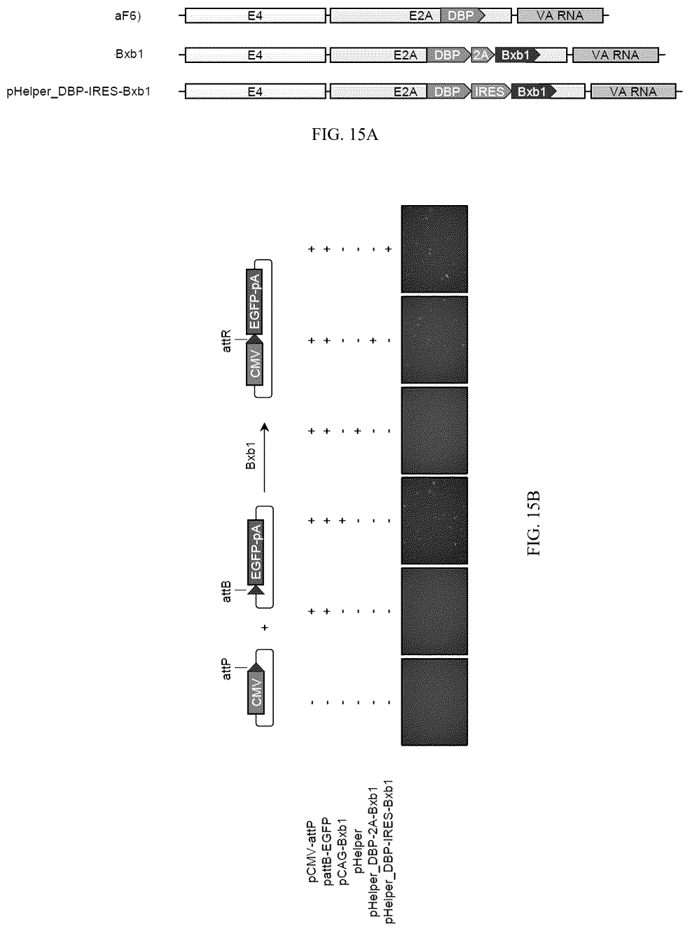

To deliver the Bxb1 gene, Bab/was inserted it downstream of the DNA-binding protein (DBP) coding sequence in pHelper via either a T2A ribosomal skipping element (pHelper_DBP-2A-Bxb1) or an internal ribosomal entry site (IRES; pHelper_DBP-IRES-Bxb1) (FIG. 15A). Both designs leverage the endogenous adenoviral DBP gene promoter and 3′ UTR, so that the entire Bxb1 expression cassette (from DBP promoter to 3′UTR) spans approximately 7 kb and greatly exceeds the AAV packaging capacity (5 kb), thus preventing the potential packaging of a functional Bxb1 gene in rAAV products. In a Bxb1-dependent reporter assay, both designs mediated successful attP/attB recombination to reconstitute reporter (i.e., enhanced green fluorescent protein, EGFP) expression (FIG. 15B), demonstrating functional Bxb1 expression.

### Example 7: AAVPure^{Mfg} Improves AAV Vector Purity

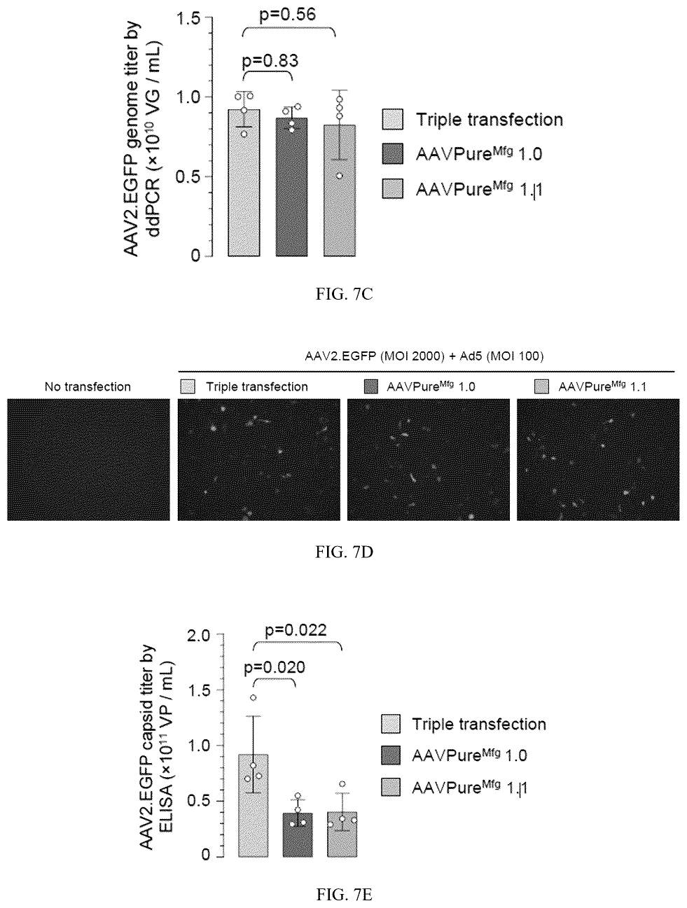

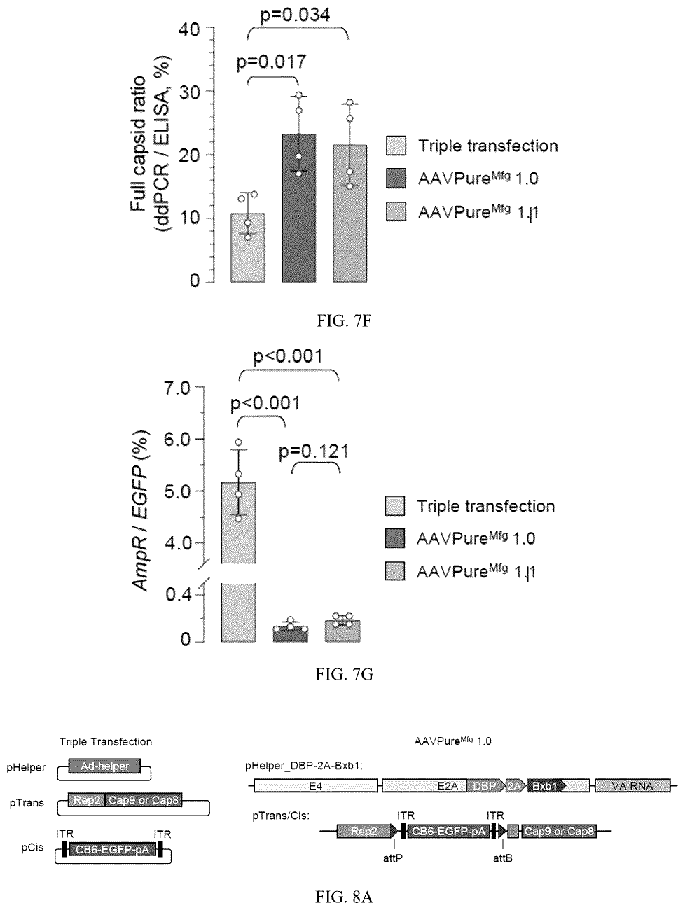

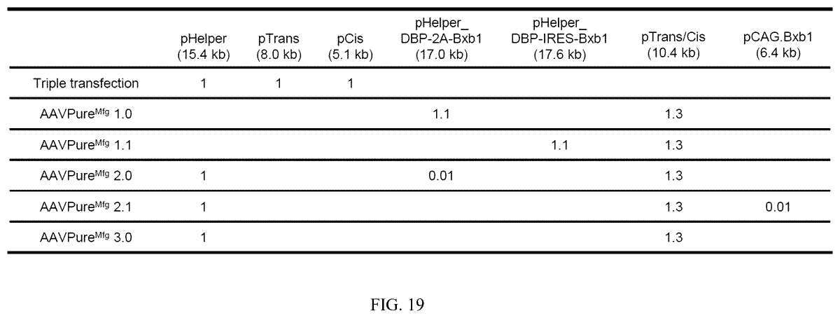

The T2A and IRES designs were designated as AAVPure^{Mfg} 1.0 and AAVPure^{Mfg} 1.1, respectively, and initially produced AAV2.EGFP vectors for characterization (FIGS. 7A and 7B). Compared with triple transfection, both AAVPure^{Mfg} 1.0 and 1.1 produced similar rAAV genome titers with approximately 20% less plasmid amount by mass (FIG. 7C and FIG. 19). The resulting AAV2.EGFP vectors transduced HEK293 cells with similar efficiency (FIG. 7D). Notably, despite comparable genome titers, the capsid titers of both AAVPure^{Mfg} vectors were reduced by half (FIG. 7E). Consequently, the full capsid ratio-calculated by normalizing the genome titer to capsid titer-increased 2-fold from 10.8% in triple transfection to 23.3% for AAVPure^{Mfg} 1.0 and 21.6% for AAVPure^{Mfg} 1.1 (FIG. 7F).

Consistent with previous reports that synthetic minicircle DNA significantly reduces plasmid backbone contamination, the abundance of encapsidated prokaryotic ampicillin resistance gene (AmpR) in rAAV products was diminished by 38-fold, from 5.2% in triple transfection to 0.14% in AAVPure^{Mfg} 1.0. Similarly, 0.19% of AmpR was observed in AAVPure^{Mfg} 1.1, representing a 28-fold reduction (FIG. 7G). Because AAVPure^{Mfg} 1.0 exhibited less AmpR contamination than AAVPure^{Mfg} 1.1, pHelper_DBP-2A-Bxb1 was further characterized and showed that it could indeed restore Rep and Cap expression from pTrans/Cis (FIG. 14B, lane 4), which enabled ITR-flanked vector genome amplification (FIG. 142C, panel 5).

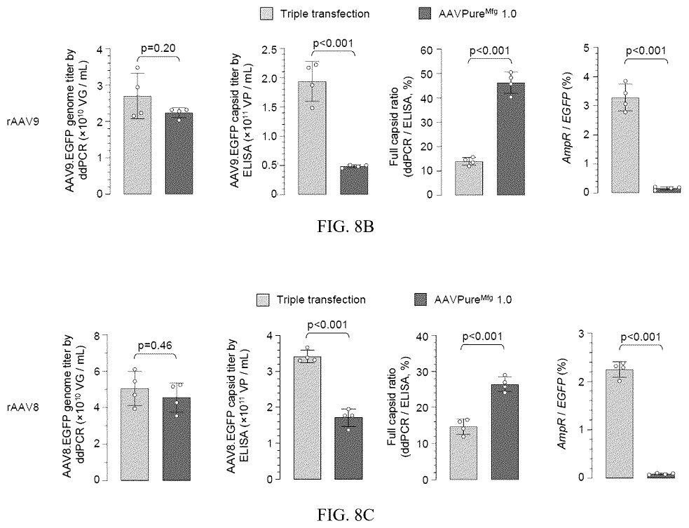

To assess the broad applicability of AAVPure^{Mfg} 1.0, this platform was tested to produce AAV9 and AAV8 vectors, two serotypes commonly used in clinical development (FIG. 8A). Consistent with our findings with AAV2, AAVPure^{Mfg} 1.0 produced similar rAAV9 and rAAV8 genome titers as compared with triple transfection, whereas the full capsid ratios increased by 2- to 3-fold with drastically reduced AmpR gene encapsidation (FIGS. 8B and 8C).

### Example 8: Mechanism of Empty Capsid Reduction

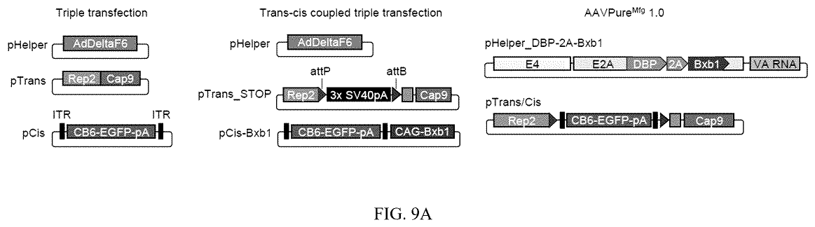

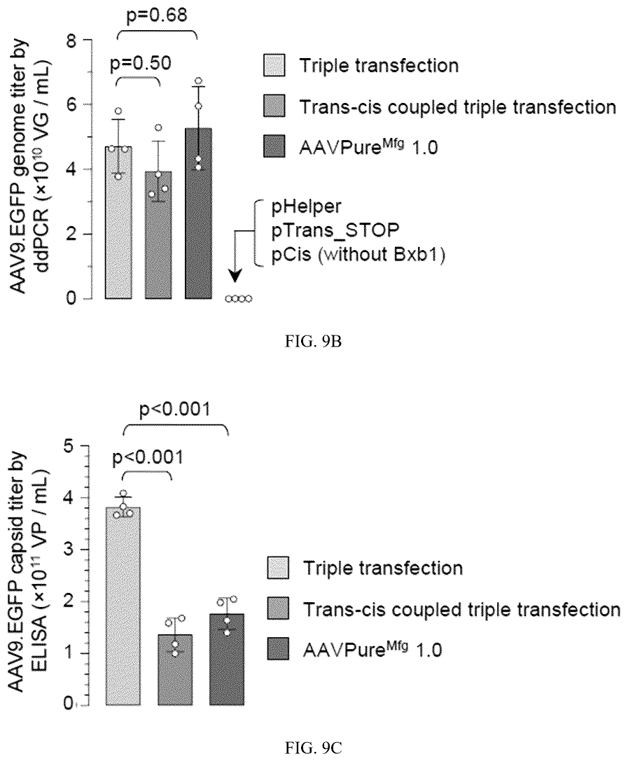

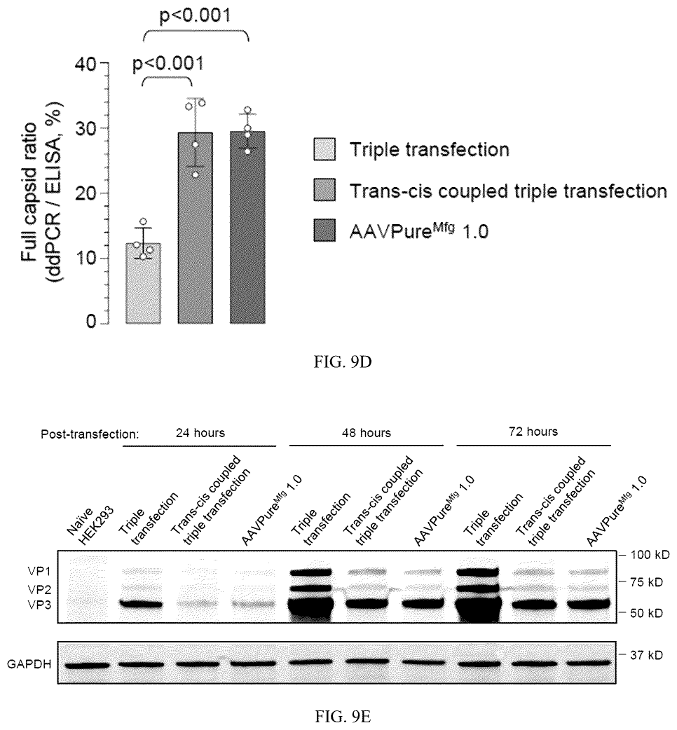

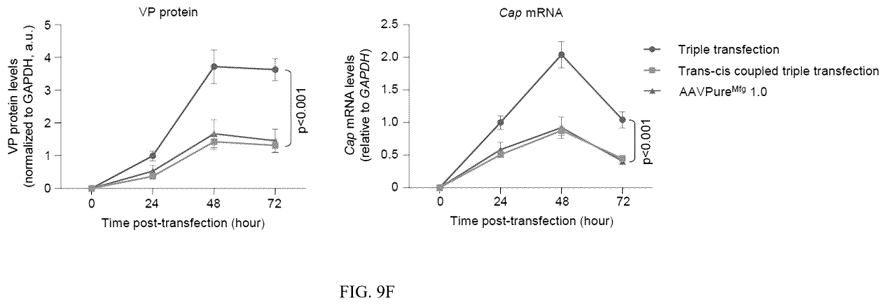

It was hypothesized that AAVPure^{Mfg} reduced empty capsid formation by eliminating the possibility of functional Rep and Cap expression in the absence of pCis, e.g., when some HEK293 cells were co-transfected by only pHelper and pTrans, but not pCis, in triple transfection (FIGS. 6A and 6B). To test this hypothesis, a Trans-Cis coupled triple transfection system consisting of three plasmids was devised (FIG. 9A, middle panel): (1) the standard pHelper, (2) pTrans-STOP that contains the attP/attB-flanked SV40 pA (three copies) in replacement of attR2, and (3) pCis-Bxb1 that provides the ITR-flanked transgene cassette and a separate Bxb1 cassette. This new system differs from standard triple transfection in that, when pHelper and pTrans-STOP coexist in a HEK293 cell nucleus without pCis-Bxb1, no capsid proteins are expressed because the SV40 pA prematurely terminates Cap transcription, thus preventing empty capsid formation. Coexistence of all three plasmids enables Bxb1-mediated excision of SV40 pA, restoring Rep and Cap expression for vector packaging. As expected, replacing pCis-Bxb1 with the standard pCis failed to produce rAAV (FIG. 9B), demonstrating the tight Rep/Cap expression regulation conferred by pTrans-STOP.

It was found that the Trans-Cis coupled triple transfection and AAVPure^{Mfg} 1.0 showed similar characteristics in rAAV production regarding genome titer, capsid titer, and full capsid ratio (FIGS. 9B-9D). The dynamics of VP and Cap mRNA expression levels in HEK293 cells were also assessed, and it was observed that both peaked at 48 hours post-transfection in all three production schemes (FIGS. 9E-9F). However, VP and Cap mRNA levels in Trans-Cis coupled triple transfection and AAVPure^{Mfg} 1.0 were 2- to 3-fold lower than those in triple transfection across all time points examined (FIGS. 9E and 9F), in agreement with the reduced capsid titers (FIG. 9C).

In summary, these results indicate that asynchronous presence of pTrans and pCis is a major contributor to empty capsid formation in standard triple transfection. Coupling pTrans and pCis through either Trans-Cis coupled triple transfection or AAVPure^{Mfg} 1.0 prevents functional Rep/Cap expression in the absence of pCis, and therefore reduces empty capsid formation and improves full capsid ratio.

### Example 9: Developing AAVPure^{Mfg} 2.0 that Utilizes Standard pHelper

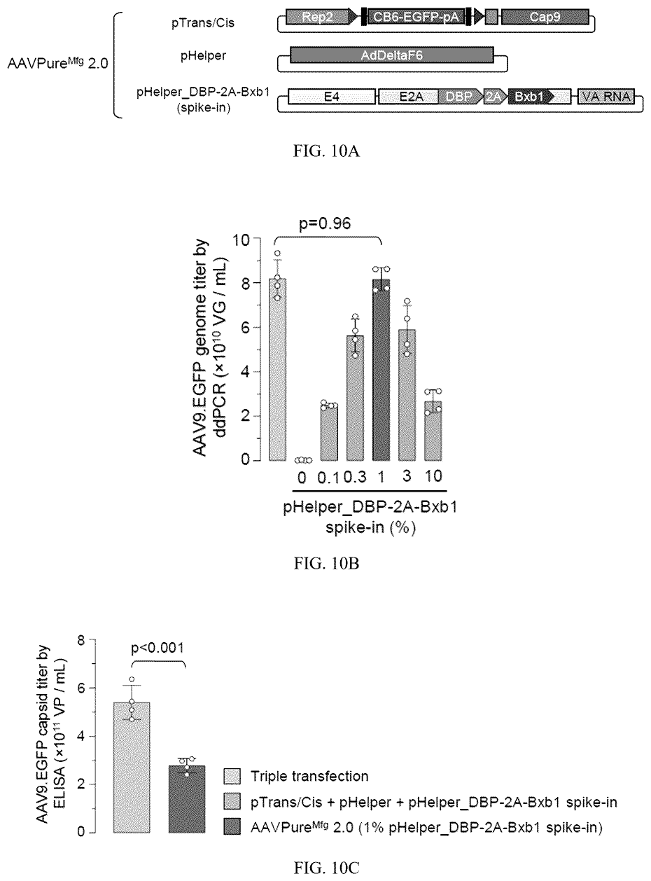

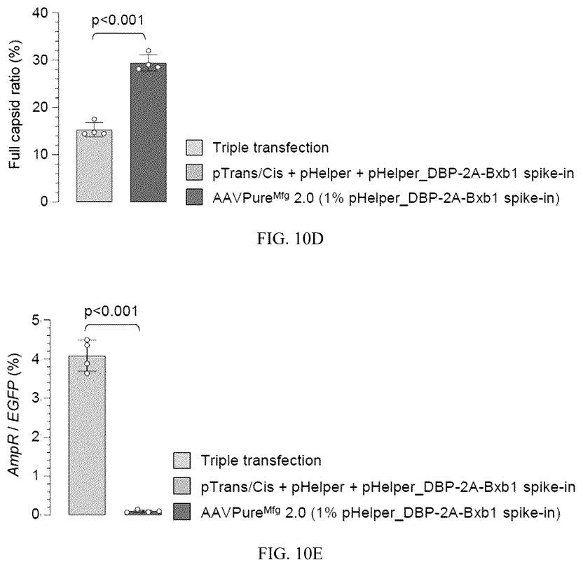

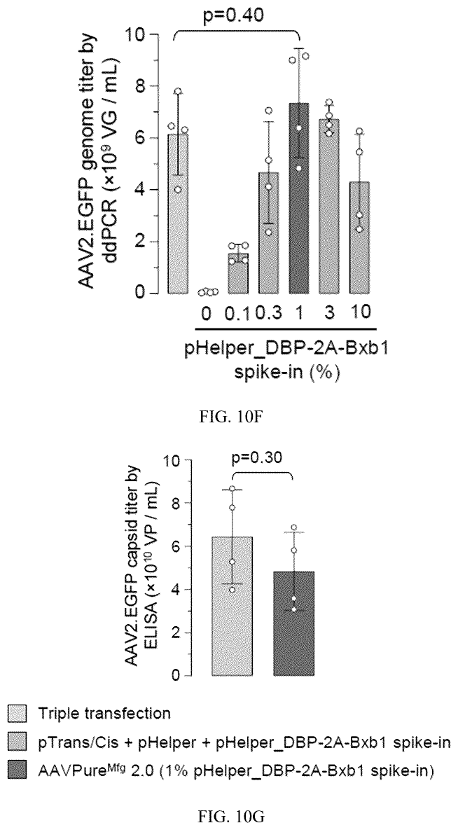

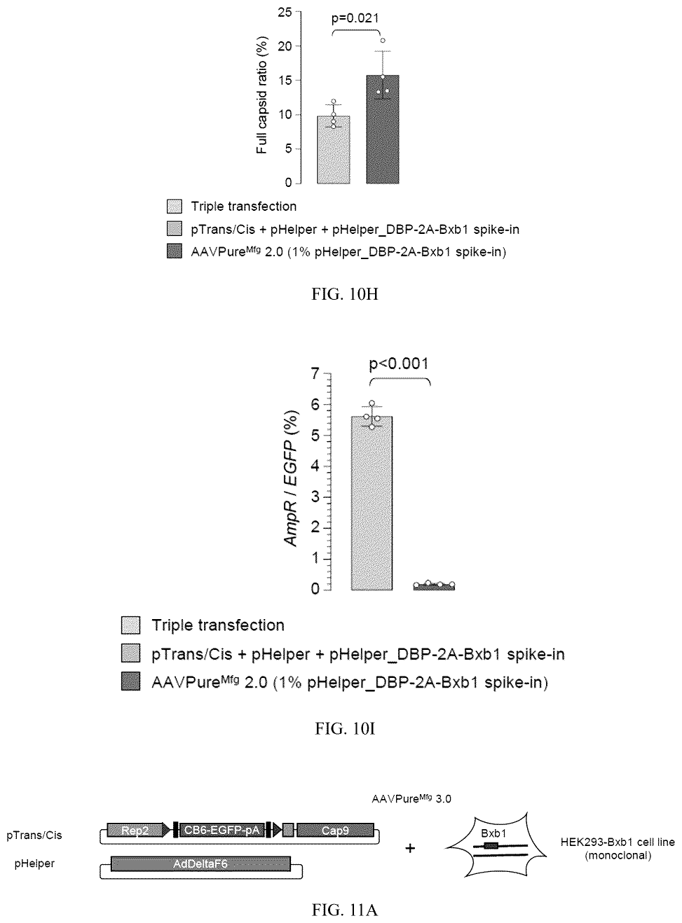

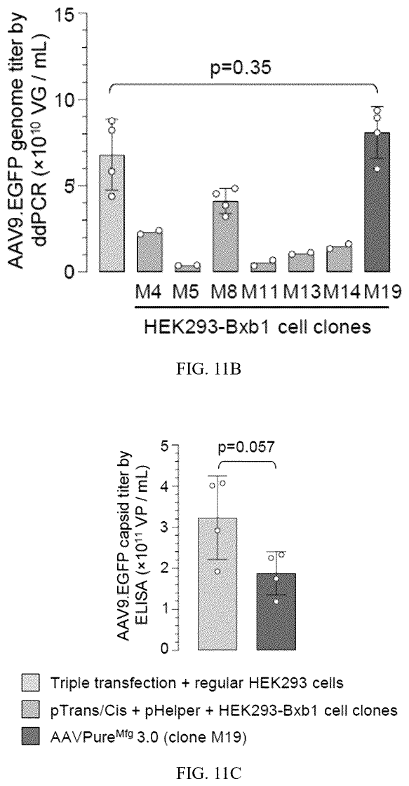

Next, to test whether AAVPure^{Mfg} is compatible with the standard pHelper, DBP-2A-Bxb1 (pHelper-Bxb1 hereafter) was supplemented with a small amount of pHelper (FIG. 10A). This design, designated as AAVPure^{Mfg} 2.0, aims to utilize the existing pHelper in various established triple or dual transfection systems, and lower the manufacturing burden for pHelper-Bxb1.

First, the optimal amount of pHelper-Bxb1 spike-in was screened for producing AAV9.EGFP. It was found that 1% (relative to the mass of standard pHelper) yielded the highest vector genome titer, which was comparable to that by triple transfection (FIG. 10B and FIG. 19). Similar to AAVPure^{Mfg} 1.0, AAVPure^{Mfg} 2.0 improved the full capsid ratio by 2-fold (FIGS. 10C and 10D), and drastically reduced plasmid backbone contamination by 40-fold compared with triple transfection (FIG. 10E). Similar results were observed when producing AAV2 vectors (FIGS. 10F-I), indicating the broad applicability of AAVPure^{Mfg} 2.0 for producing high-purity AAV vectors.

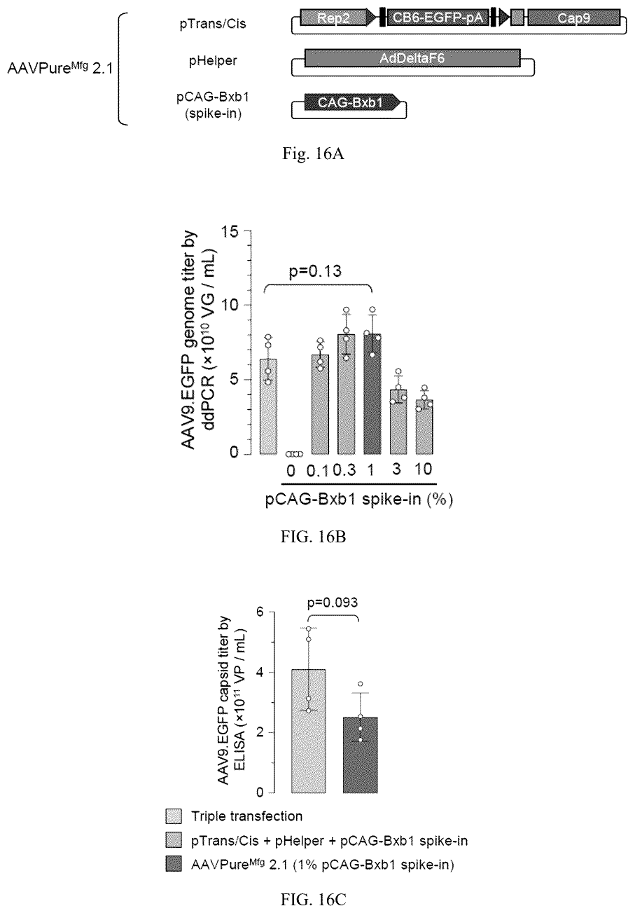

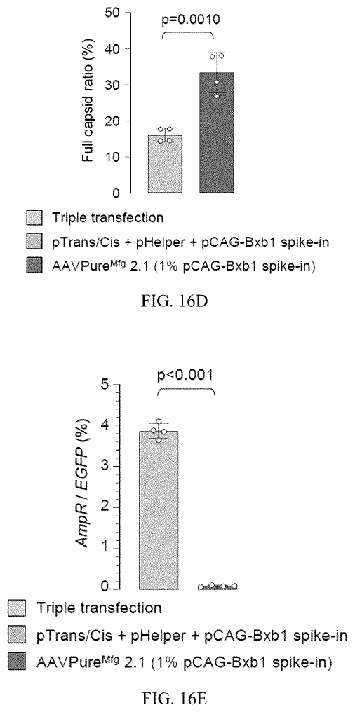

Next, whether other Bxb1-expressing plasmids of smaller sizes could replace the large pHelper-Bxb1 was investigated. To this end, pCAG-Bxb1 (Addgene, 51271) as the spike-in was used (FIG. 16A and FIG. 19). It was found that as little as 0.1% pCAG-Bxb1 spike-in led to efficient rAAV production (FIG. 16B), as compared to pHelper-Bxb1 that performed the best when supplemented at 1% (FIG. 16A). This was likely due to its smaller size that led to more efficient transfection, or the strong CAG promoter that drove higher Bxb1 expression. Nevertheless, using 1% pCAG-Bxb1 spike-in was designated as AAVPure^{Mfg} 2.1, because it also afforded efficient rAAV production (FIG. 16B). Consistent with previous AAVPure^{Mfg} iterations, rAAV produced by AAVPure^{Mfg} 2.1 showed a 2-fold increase in full capsid ratio (FIGS. 16C-16D) and a 45-fold decrease in prokaryotic DNA contamination (FIG. 16E).

### Example 10: a Stable HEK293-Bxb1 Cell Line Enables the Development of AAVPure^{Mfg} 3.0

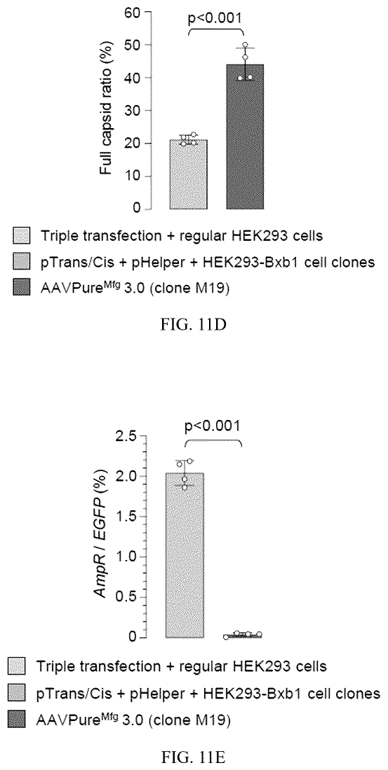

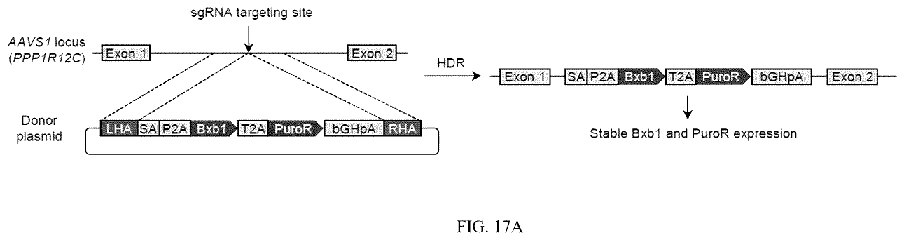

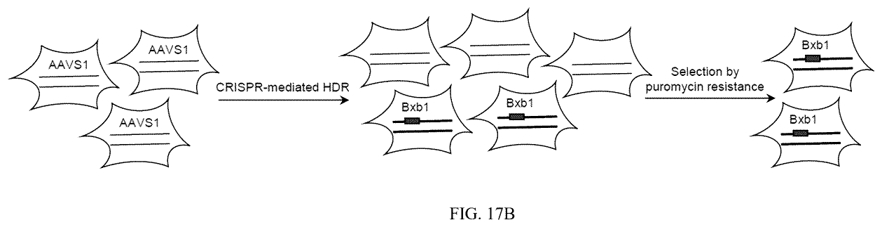

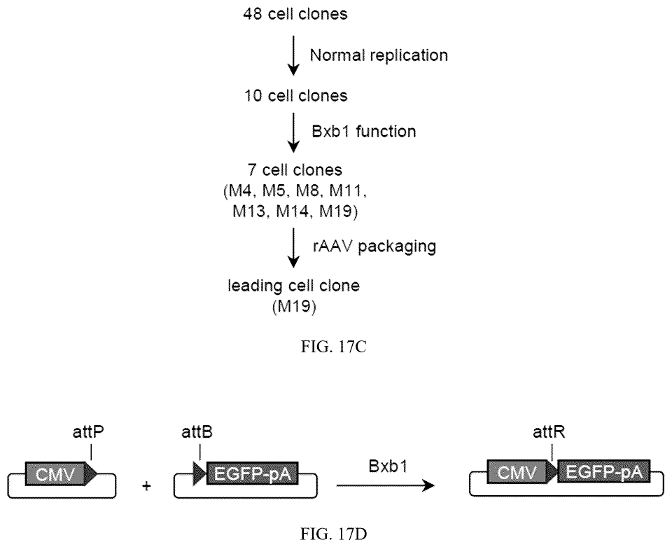

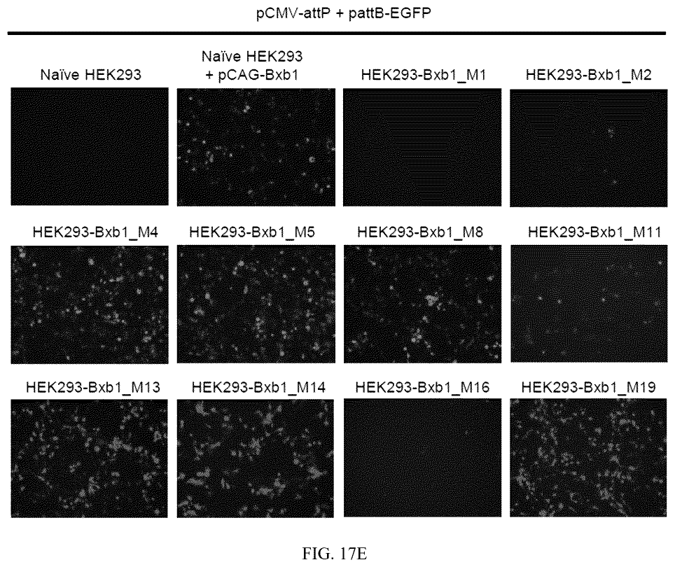

The compatibility with various Bxb1-expressing plasmids prompted us to develop AAVPure^{Mfg} 3.0 consisting of a stable monoclonal HEK293-Bxb1 cell line (FIG. 11A). This approach obviates Bxb1 plasmid usage, thus further decreasing rAAV manufacturing costs. Using a CRISPR-mediated homology directed repair (HDR) strategy, Bxb1 was inserted into the AAVS1 safe harbor locus known to enable robust stable transgene expression. The HDR template includes an artificial splicing acceptor (SA) and P2A/T2A ribosomal skipping elements, so that both Bxb1 and a puromycin resistance gene (PuroR) are under control of the endogenous AAVS1 promoter, thereby avoiding the introduction of an exogenous promoter that is often subjected to silencing (FIGS. 17A and 17B). 48 puromycin-resistant cell clones were obtained, 10 of which showed growth rates comparable to that of unmodified HEK293 cells; further analysis identified seven clones what exhibited robust Bxb1 recombinase activity (FIGS. 17C-E).

Next, the seven candidate HEK293-Bxb1 cell clones were evaluated for producing AAV9.EGFP following the AAVPure^{Mfg} 3.0 scheme (FIG. 6A). It was found that clone M19 generated the highest rAAV genome titer among all cell clones, which was comparable to that produced by standard triple transfection using unmodified HEK293 cells (FIG. 6B). AAVPure^{Mfg} 3.0 using M19 improved full capsid ratio by 2-fold, and decreased prokaryotic DNA encapsidation by 51-fold (FIGS. 11C-11E).

### Example 11: AAVPure^{Mfg} with Suspension HEK293 Cells

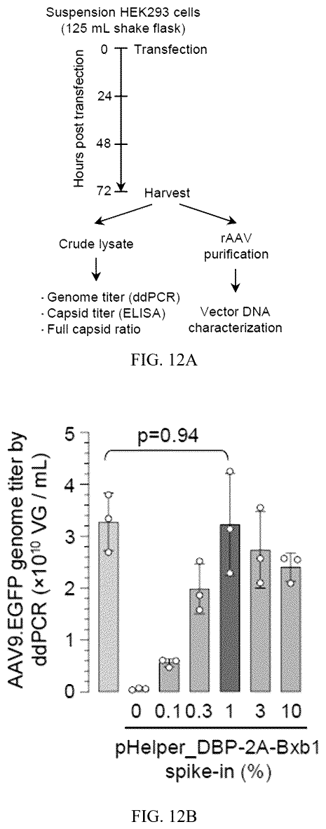

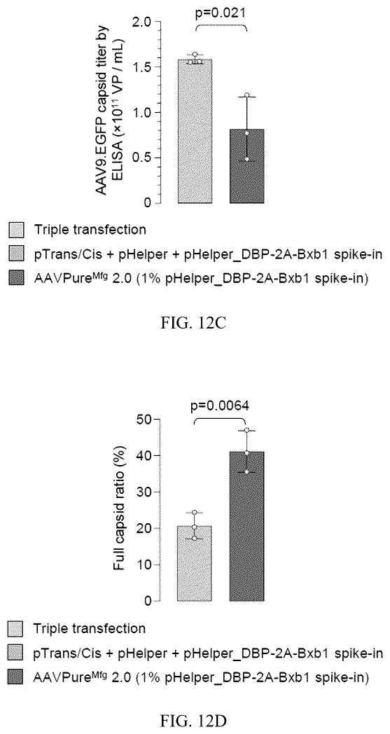

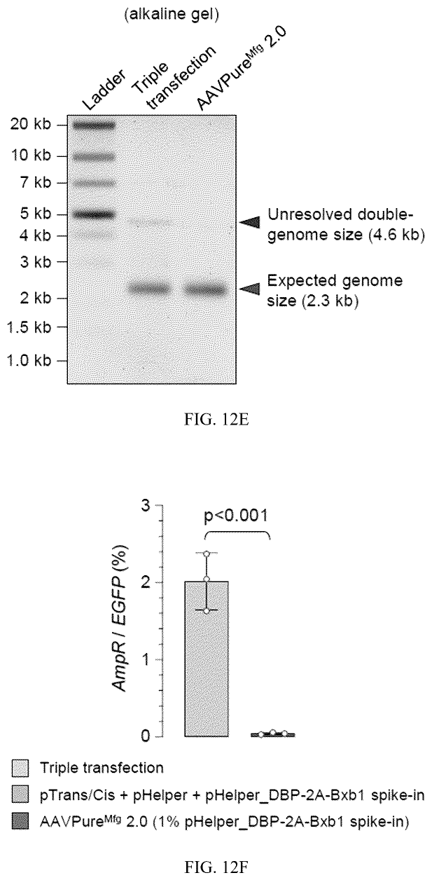

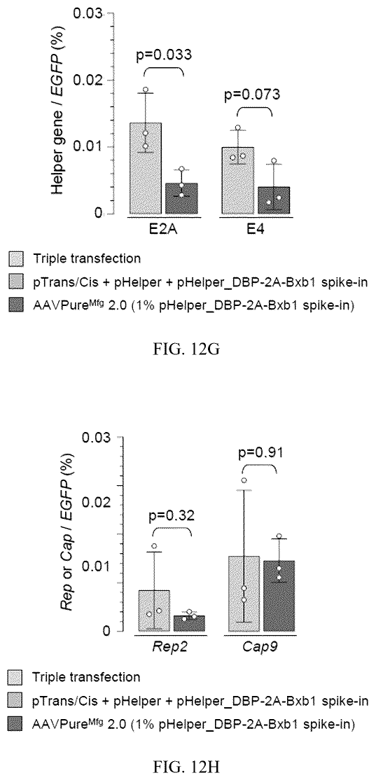

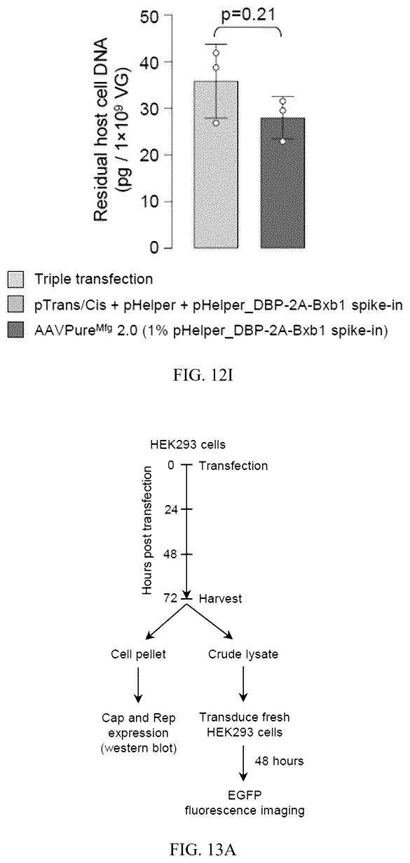

Next, AAVPure^{Mfg} was tested using suspension HEK293 cells, a platform that is commonly used in rAAV manufacturing at scale including at cGMP level (FIG. 12A). Consistent with the results obtained with adherent HEK293 cells, AAVPure^{Mfg} 2.0 (i.e., with 1% pHelper-Bxb1 spike-in) produced similar AAV9.EGFP titer as standard triple transfection in suspension HEK293 cells (FIG. 12B). In contrast, the capsid titer was reduced by half, leading to a 2-fold increase in full capsid ratio from 20.7% in triple transfection to 41.1% in AAVPure^{Mfg} 2.0 (FIGS. 12C-12D).

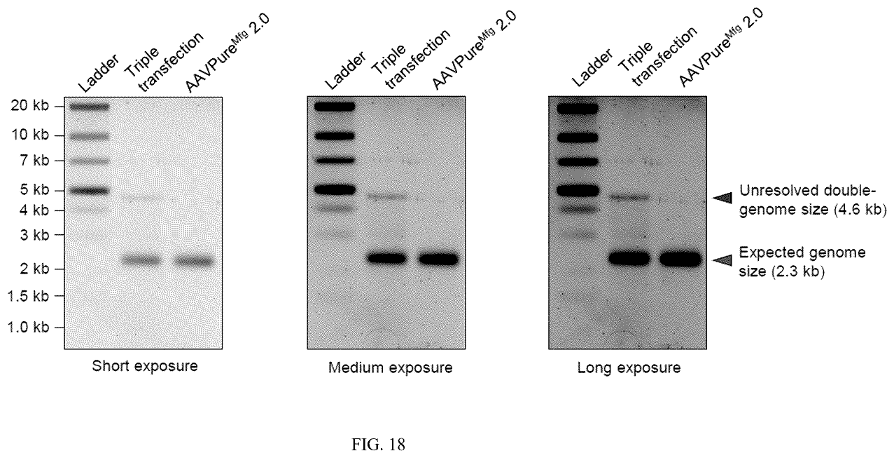

For rAAV generated by both methods, the vector DNA was purified to evaluate their size distribution by denaturing alkaline gel electrophoresis. Both vectors contained a 2.3 kb DNA species of expected vector genome size; however, a larger band corresponding to the double-sized genome (4.6 kb) was detectable only in the vector produced by triple transfection (FIG. 12E and FIG. 18), likely due to incomplete ITR resolution as previously reported for other rAAV preparations. These data indicate that AAVPure^{Mfg} 2.0 produces rAAV with improved vector genome homogeneity. Various DNA impurities in purified rAAV were quantified by targeted droplet digital PCR (ddPCR). As demonstrated using adherent HEK293 cells, plasmid backbone DNA in the AAVPure^{Mfg} 2.0 vector showed a 45-fold reduction as compared with the triple transfection vector (FIG. 12F). The AAVPure^{Mfg} 2.0 vector contained lower or equal amount of other known DNA impurities, including the adenoviral helper genes E2A and E4, AAV Rep and Cap genes, and host cell DNA (FIGS. 12G-12I). Together, these data demonstrate that AAVPure^{Mfg} with suspension HEK293 cells effectively produces rAAV with improved full capsid ratio and vector DNA purity.

### Example 12: High-Purity AAV Vector Production Utilizing Recombination-Dependent Minicircle Formation and Genetic Coupling

Aspects, of this disclosure disclose minicircle dual transfection also known as AAVPure^{Mfg}, an improved rAAV manufacturing platform that addresses two critical limitations inherent to triple transfection: high levels of plasmid backbone contaminants and empty capsid formation. AAVPure^{Mfg} builds on a series of engineering steps that synergize Bxb1 recombinase, minicircle DNA, and the structural flexibility of Rep C-terminus domain. This is the first demonstration that a minicircle cis construct can be generated from a parental plasmid during rAAV production phase, and that this strategy dramatically reduces plasmid backbone encapsidation to around 0.1%, an unprecedently low level in plasmid-based rAAV manufacturing. This pronounced improvement is expected to markedly enhance the safety profile of rAAV products. AAVPure^{Mfg} boosts full capsid ratios up to 3-fold at harvest. These results reveal that, besides the temporal desynchronization between capsid synthesis and vector genome replication, spatial desynchronization between pTrans and pCis plays an important role in empty capsid formation in triple transfection. An improved full capsid ratio in cell lysate is expected to greatly facilitate downstream purification, thus reducing manufacturing costs and enhancing vector potency. Finally, the data demonstrates that AAVPure^{Mfg} is broadly applicable across different serotypes and cell culture systems.

Recently, Lieshout et al. (Mol Ther Methods Clin Dev 29, 426-436) developed a dual-plasmid system, pOXB, which combines pTrans and pCis into a single plasmid by inserting an ITR-flanked transgene cassette next to Rep/Cap in pTrans. While this approach improves the full capsid ratio presumably due to the physical linkage of Rep/Cap and cis construct, it appears to do so to a lesser extent than AAVPure^{Mfg}. Furthermore, the pOXB design still suffers from potential prokaryotic backbone encapsidation and risks generating replication-competent AAV (rcAAV) due to the close proximity between functional Rep/Cap genes and ITRs. In contrast, the pTrans/Cis used in AAVPure^{Mfg} features an ITR-flanked transgene embedded within Rep/Cap genes and abolishing their expression and function at the default stage, thereby mitigating the chance of generating rcAAV.

Besides the initial AAVPure^{Mfg} design, AAVPure^{Mfg} 2.0 utilizes the same pHelper as for triple transfection and a small amount of modified pHelper expressing Bxb1. As pHelper is universally used in various plasmid-based AAV manufacturing systems, this iteration is expected to greatly reduce the manufacturing burden for pHelper-Bxb1. Interestingly, various Bxb1-expressing plasmids are compatible with AAVPure^{Mfg}, which further facilitates its implementation. However, it is noted that it is possible for a small Bxb1 expression cassette, such as the CAG-Bxb1 (3.5 kb) used in AAVPure^{Mfg} 2.1, to be packaged into rAAV and expressed in recipients. From this perspective, the pHelper-Bxb1 used in AAVPure^{Mfg} 2.0 is designed to be much safer, because the entire Bxb1 cassette is around 7 kb, far beyond the AAV packaging capacity. This concern is further mitigated in AAVPure^{Mfg} 3.0, where Bxb1 is stably integrated into the AAVS1 safe harbor locus in HEK293 cells and driven by the endogenous promoter.

An unexpected finding from this study is the improved vector genome homogeneity in AAVPure^{Mfg}, evidenced by the reduction of double-sized vector genome that is commonly observed in rAAV preparations (FIG. 12E and FIG. 18). While the precise mechanism remains to be studied, one possibility is that genetic coupling of pTrans and pCis in AAVPure^{Mfg} enhances the accessibility of vector genome to Rep proteins, increasing the likelihood of ITR resolution. Another interesting finding is that the Rep coding region between P40 and intron is amenable to in-frame peptide insertion. The plasticity of this Rep region in the context of rAAV production is also described in a recent Rep saturation mutagenesis screen. Future work may exploit this unique region to engineer Rep protein variants as valuable tools for AAV biology studies.

In summary, AAVPure^{Mfg} produces high-purity AAV vectors with markedly improved full capsid ratios, better vector genome homogeneity, and dramatically reduced prokaryotic DNA contaminants. Together with reduced plasmid demand, AAVPure^{Mfg} provides a valuable platform to manufacture rAAV and can potentially make high-quality AAV genetic medicines more readily accessible to patients.

### Example 13: Materials and Methods

### Plasmid Construction

Plasmids were constructed using Gibson Assembly. The plasmids pAAV2/2-attR1 (pHL280b), pAAV2/2-attR2 (pHL281b), and pAAV2/2-attR3 (pHL282b) were generated by digesting the packaging plasmid pAAV2/2 with HindIII and subsequently assembling the digestion products with corresponding gBlocks from Integrated DNA Technologies (IDT), using NEBuilder HiFi DNA Assembly Master Mix (NEB, E2621L). For the construction of pHelper_DBP-2A-Bxb1 (pHL284) and pHelper_DBP-IRES-Bxb1 (pHL285), a NsiI digestion site was introduced following the stop codon of DBP in the pHelper plasmid (Addgene, 112867). The 2A-Bxb1 and IRES-Bxb1 DNA fragments were then assembled via Gibson Assembly, with the 2A and IRES fragments carried in IDT gBlocks. The Bxb1 gene was amplified via PCR from pCAG-Bxb1 (Addgene, 51271) using Phusion High-Fidelity DNA Polymerase (NEB, M0530S). pTrans/Cis plasmids were generated by digestion of pCis plasmid pAAV.CB6-EGFP with PacI, and then assembling this ITR-flanked transgene DNA with pAAV2/2, pAAV2/8, or pAAV2/9 packaging plasmids by Gibson Assembly at the attR2 site. Similar strategies were used to generate pTrans_STOP (pAAV2/9-3XSV40 pA, pHL327) with the 0.8 kb 3XSV40 pA fragment obtained from HindIII and BamHI digestion of pAAV.CAG.LSL.EGFP (Addgene, 100047). The left homologous arm (LHA)-splicing acceptor (SA)-P2A, T2A-PuroR-bGH pA, and right homologous arm (RHA) fragments in donor plasmid of pAAVS1 LHA-SA-P2A-BxB1-T2A-PuroR-bGH pA-RHA (pHL380) were ordered from IDT as gBlocks. Bxb1 DNA fragment was also generated through PCR from pCAG-Bxb1 (Addgene, 51271) by Phusion High-Fidelity DNA Polymerase (NEB, M0530S). These fragments were then assembled into donor plasmid by Gibson Assembly. The detailed plasmid sequences can be found in Table S2.

### Cell Culture

Adherent HEK293 cells (CRL-1573) were obtained from ATCC and maintained in DMEM (Gibco, 11965-084) supplemented with 10% (v/v) Fetal Bovine Serum (FBS; Gibco, 26140-079) and 1% (v/v) penicillin/streptomycin (Thermo Fisher Scientific, 15140122) at 37° C. in a humidified atmosphere containing 5% CO2. Suspension Expi293F cells, purchased from Thermo Fisher Scientific (A14528), were cultured in Freestyle F17 media (Gibco, A13835) supplemented with 10 mM Glutamax (Gibco, 35050061) at 37° C. with 5% CO2, 80% humidity, and shaking at 120 rpm.

### Monoclonal HEK293-Bxb1 Cell Line Generation

For the generation of the HEK293-Bxb1 cell line, adherent HEK293 cells were transfected using a mixture of a Cas9-expressing plasmid, an AAVS1-targeting sgRNA plasmid (from Dr. Alex Brown), and the donor plasmid pAAVS1 LHA-SA-P2A-BxB1-T2A-PuroR-bGH (pHL380) at a mass ratio of 1:1:3. Transfection was performed using Lipofectamine 2000 (Thermo Scientific, 11668027). Three days post-transfection, the cells were split and selected with puromycin (1 μg/mL) for 7 days. Monoclonal colonies were isolated through serial dilution and confirmed via a Bxb1 function assay.

### Aav Vector Production Using Adherent HEK293 Cells

Small-scale vector preparations were generated in 12-well plates. In triple transfection, HEK293 cells were transfected with three plasmids carrying the vector genome (pCis), Rep/Cap (pTrans, pRep2/CapX; Addgene #112865) and adenovirus helper genes (pHelper; Addgene #112867), respectively, at equal mass ratio of 1:1:1 using calcium phosphate method (Promega, E1200), totaling 1.5 μg/well. In AAVPure^{Mfg}, the detailed plasmid sequence of pHelper-Bxb1 and pTrans/Cis plasmids can be found in Table S2. pCAG-Bxb1 was purchased from Addgene (Plasmid #51271). pHelper-Bxb1 and pTrans/Cis were transfected with equal mole as that of pHelper and pTrans in HEK293 cells as described in Table S1. Transfected cells were maintained in DMEM supplemented with 10% FBS and 1% penicillin/streptomycin for 24 hours, after which the culture medium was replaced with DMEM supplemented with 1% penicillin/streptomycin without FBS. 72 hours post transfection, cells and culture media were harvested, and subjected to 3 successive freeze-thaw cycles. The crude lysates were centrifuged at 14,000 g/min for 15 min at 4° C. to remove cell debris. Cleared crude lysates were treated with DNase-I and protease K, and used in a droplet digital PCR (ddPCR) assay to determine titer.

### Aav Vector Production Using Suspension Expi293F Cells.

30 mL Expi293F cells (Thermo Scientific, A14528) cultured in a 125 mL shaker flask were transfected at the density of 1×10⁶ cells/mL using PEI Max (PolySciences, 24765-2). For triple transfection, three plasmids were co-transfected: one carrying the vector genome (pCis, 10 μg), one carrying Rep/Cap (pTrans, pRep2/CapX, 10 μg), and one carrying adenovirus helper genes (pHelper, 10 μg). For AAVPure^{Mfg} 2.0, the three plasmids were pHelper (10 μg), pTrans/Cis (13 μg) and spike-in pHelper_DBP-2A-Bxb1 (ranging from 0.01 μg (0.01%) to 1 ug (10%) as indicated). Transfected cells were maintained in Freestyle F17 media supplemented with 10 mM Glutamax. 72 hours post transfection, a small aliquot of cells (1 mL) was used to prepare cleared lysate for determining genome and capsid titers. The remaining cells (29 mL) were purified using the AAVpro Purification Kit (Takara Bio, 6675) for vector DNA characterization.

Quantification of Vector DNA Impurities by ddPCR

rAAV crude lysates or purified AAV vectors were treated with DNase-I (Roche Life Science, 4716728001) and protease K (QIAGEN, 19133). Duplexing Taqman ddPCR assays were performed with one reagent targeting EGFP transgene (Thermo Fisher Scientific, Mr00660654_cn), and the other targeting AmpR, adenoviral helper genes, or AAV Rep/Cap genes. The vector DNA impurity was calculated by normalizing its value to that of EGFP.

```
AmpR plasmid backbone Taqman reagent:
  (SEQ ID NO: 12)
Forward primer: 5′-GATAAATCTGGAGCCGGTGAG;
  (SEQ ID NO: 13)
reverse primer: 5′-AGATAACTACGATACGGGAGGG;
  (SEQ ID NO: 14)
probe: 5′-TGGGTCTCGCGGTATCATTGCAG.
pHelper E2A Taqman reagent:
(SEQ ID NO: 15)
Forward primer: 5′-CTGGGCTCTTCCTCTTCCTC;
  (SEQ ID NO: 16)
reverse primer: 5′-GATGTGGCGCTACAAATGGT;
  (SEQ ID NO: 17)
probe: 5′-TTCAGCCGCCGCACTGTGCG.
pHelper E4 Taqman reagent:
(SEQ ID NO: 18)
Forward primer: 5′-AAGACCTCGCACGTAACTCA;
  (SEQ ID NO: 19)
reverse primer: 5′-GGCATGACACTACGACCAAC;
(SEQ ID NO: 20)
probe: 5′-CGGTTGTCTCGGCGCACTCCG
pTrans Rep2 Taqman reagent:
(SEQ ID NO: 21)
Forward primer: 5′-CTCAACGACCTTCGAACACC;
  (SEQ ID NO: 22)
reverse primer: 5′-ACCTCAACCACGTGATCCTT;
  (SEQ ID NO: 23)
probe: 5′-CCGGTCTTGCAACGGCTGCT.
pTrans Cap9 Taqman reagent:
(SEQ ID NO: 24)
Forward primer: 5′-ATGGACAAGTGGCCACAAAC;
  (SEQ ID NO: 25)
reverse primer: 5′-ATCAGCGGAGAAGGGTGAAA;
  (SEQ ID NO: 26)
probe: 5′-CAGCCGGTCTGCGCCTGTGC.
```

Quantification of rAAV Full Capsid Ratio

rAAV genome titer (vg/mL) in crude lysate was quantified by ddPCR using a Taqman reagent targeting the EGFP transgene (Thermo Fisher Scientific, Mr00660654_cn) post treatment with DNase-I (Roche Life Science, 4716728001) and protease K (QIAGEN, 19133). rAAV particle concentration (pt/mL) in crude lysate was quantified using the AAV2 Xpress ELISA Kit (PROGEN Biotechnik, PRAAV2XP), AAV8 Xpress ELISA Kit (PROGEN Biotechnik, PRAAV8XP), or AAV9 Xpress ELISA Kit (PROGEN Biotechnik, PRAAV9XP) following manufacturer's manual. Full capsid ratio is calculated as genome titer divided by capsid titer.

### Western Blotting

Cultured cells were pelleted by centrifugation at 1,000 g/min for 5 min at 4° C., and then lysed with M-PER (Thermo Fisher Scientific, 78501) with protease inhibitor (Roche, 4693159001). Protein concentration was determined using Pierce BCA Protein Assay Kit (Pierce, 23225). Normalized protein lysates were boiled for 10 min in reducing SDS sample buffer (Boston BioProducts, BP-111R). Primary antibodies: mouse anti-Rep (Origen Technologies, AM09104PU-N, 1:100), mouse anti-VP1/2/3 (PROGEN Biotechnik, 61058, 1:200), rabbit anti-GAPDH (Abcam, ab9485, 1:2000). Secondary antibodies: LICOR IRDye 680RD goat anti-mouse IgG (H+L) (LI-COR Biosciences, 926-68070, 1:3000), LICOR IRDye 800CW goat anti-rabbit IgG (H+L) (LI-COR Biosciences, 926-32211, 1:3000). Blot membranes were imaged by LI-COR scanner (Odyssey) and quantified by ImageJ Fiji.

### AAV Vector DNA Analysis by Alkaline Agarose Gel Electrophoresis

0.8% agarose gel was prepared by boiling agarose in ultra-pure water, followed by cooling to 55° C. and adding 0.1 volume of 10× alkaline gel electrophoresis buffer (500 mM NaOH and 10 mM EDTA). 200 μl of purified rAAV was treated with DNase-I and protease K, and then purified by phenol:chloroform:isoamyl alcohol solution. Purified vector DNA was mixed with 6× alkaline gel loading buffer (Thermo Fisher Scientific, AAJ62157AB), and loaded to alkaline gel. Electrophoresis was performed at a voltage of 3V/cm for approximately 3 hours. Then the gel was soaked in neutralization solution (BioWorld, 10750014) for 1 hour at room temperature. The neutralized gel was stained with SYBR Gold (1:10,000 dilution, Thermo Fisher Scientific, S-11494) in 1×TAE buffer for 15 min, and imaged using a Bio-Rad Gel Doc XR+ Imaging System.

### Statistical Analysis

Data were presented as mean±standard deviation (SD). Comparison among two groups was analyzed by unpaired t test. Comparison among multiple groups was analyzed by one-way analysis of variance (ANOVA) followed by Dunnett's multiple comparisons test. GraphPad Prism 10 was used for statistical analysis and data plotting.

```
Sequences:
Bxb1:
  (SEQ ID NO: 1)
MRALVVIRLSRVTDATTSPERQLESCQQLCAQRGWDVVGVAEDLDVSGAVDPFDRKR
RPNLARWLAFEEQPFDVIVAYRVDRLTRSIRHLQQLVHWAEDHKKLVVSATEAHFDTT
TPFAAVVIALMGTVAQMELEAIKERNRSAAHFNIRAGKYRGSLPPWGYLPTRVDGEWR
LVPDPVQRERILEVYHRVVDNHEPLHLVAHDLNRRGVLSPKDYFAQLQGREPQGREWS
ATALKRSMISEAMLGYATLNGKTVRDDDGAPLVRAEPILTREQLEALRAELVKTSRAK
PAVSTPSLLLRVLFCAVCGEPAYKFAGGGRKHPRYRCRSMGFPKHCGNGTVAMAEWD
AFCEEQVLDLLGDAERLEKVWVAGSDSAVELAEVNAELVDLTSLIGSPAYRAGSPQRE
ALDARIAALAARQEELEGLEARPSGWEWRETGQRFGDWWREQDTAAKNTWLRSMNV
RLTFDVRGGLTRTIDFGDLQEYEQHLRLGSVVERLHTGMS
pRep2/Cap2 (Region between P40 and Intron in pTrans of pRep2/Cap2 packaging plasmid)
  (SEQ ID NO: 2)
gttgcgcagccatcgacgtcagacgcggaagcttcgatcaactacgcagacag
pRep2-attR1/Cap2 (pHL280b):
(SEQ ID NO: 3)
gttGTGGTTTGTCTGGTCAACCACCGCGGaCTCCGTCGTCAGGATCATtcagttgcgcagccatc
gacgtcagacgcggaagcttcgatcaactacgcagacag
pRep2-attR2/Cap2 (pHL281b):
  (SEQ ID NO: 4)
gttgcgcagccatcgacgtcaGTGGTTTGTCTGGTCAACCACCGCGGaCTCCGTCGTCAGGATCA
Ttcagacgcggaagcttcgatcaactacgcagacag
pRep2-attR3/Cap2 (pHL282b):
  (SEQ ID NO: 5)
gttgcgcagccatcgacgtcagacgcggaagcttcgGTGGTTTGTCTGGTCAACCACCGCGGaCTCCGTC
GTCAGGATCATatcaactacgcagacag
pHelper_DBP-2A-Bxb1 (pHL284):
(SEQ ID NO: 6)
ggcagaacccctttgattttGGATCCGGTGAGGGCAGAGGAAGTCTACTAACATGCGGTGACGT
GGAGGAGAATCCGGGCCCTatgccaaaaaagaaaagaaaagtgtatccctatgatgtccccgattatgccggttcaaga
gccctggtcgtgattagactgagccgagtgacagacgccaccacaagtcccgagagacagctggaatcatgccagcagctctgtgctca
gcggggttgggatgtggtcggcgtggcagaggatctggacgtgagcggggccgtcgatccattcgacagaaagaggaggcccaacct
ggcaagatggctcgctttcgaggaacagccctttgatgtgatcgtcgcctacagagtggaccggctgacccgctcaattcgacatctccag
cagctggtgcattgggctgaggaccacaagaaactggtggtcagcgcaacagaagcccacttcgatactaccacaccttttgccgctgtgg
tcatcgcactgatgggcactgtggcccagatggagctcgaagctatcaaggagcgaaacaggagcgcagcccatttcaatattagggccg
gtaaatacagaggctccctgcccccttggggatatctccctaccagggtggatggggagtggagactggtgccagaccccgtccagaga
gagcggattctggaagtgtaccacagagtggtcgataaccacgaaccactccatctggtggcacacgacctgaatagacgcggcgtgctc
tctccaaaggattattttgctcagctgcagggaagagagccacagggaagagaatggagtgctactgcactgaagagatctatgatcagtg
aggctatgctgggttacgcaacactcaatggcaaaactgtccgggacgatgacggagcccctctggtgagggctgagcctattctcacca
gagagcagctcgaagctctgcgggcagaactggtcaagactagtcgcgccaaacctgccgtgagcaccccaagcctgctcctgagggt
gctgttctgcgccgtctgtggagagccagcatacaagtttgccggcggagggcgcaaacatccccgctatcgatgcaggagcatggggtt
ccctaagcactgtggaaacgggacagtggccatggctgagtgggacgccttttgcgaggaacaggtgctggatctcctgggtgacgctga
gcggctggaaaaagtgtgggtggcaggatctgactccgctgtggagctggcagaagtcaatgccgagctcgtggatctgacttccctcatc
ggatctcctgcatatagagctgggtccccacagagagaagctctggacgcacgaattgctgcactcgctgctagacaggaggaactgga
gggcctggaggccaggccctctggatgggagtggcgagaaaccggacagaggtttggggattggtggagggagcaggacaccgcag
ccaagaacacatggctgagatccatgaatgtccggctcacattcgacgtgcgcggtggcctgactcgaaccatcgattttggcgacctgca
ggagtatgaacagcacctgagactggggtccgtggtcgaaagactgcacactgggatgtcctaaacccttgccgtctgcgccgt
pTrans-STOP (pHL327):
  (SEQ ID NO: 7)
gttgcgcagccatcgacgtcaGTGGTTTGTCTGGTCAACCACCGCGGaCTCAGTGGTGTACGGTA
CAAACCCAttaattaaggccttaattaggctgcgcgaagcttgcagatctgcgactctagaggatctgcgactctagaggatcataat
cagccataccacattttgtagaggttttacttgctttaaaaaacctcccacacctccccctgaacctgaaacataaaatgaatgcaattgttgttg
ttaacttgtttattgcagcttataatggttacaaataaagcaatagcatcacaaatttcacaaataaagcatttttttcactgcattctagttgtggttt
gtccaaactcatcaatgtatcttatcatgtctggatctgcgactctagaggatcataatcagccataccacatttgtagaggttttacttgctttaa
aaaacctcccacacctccccctgaacctgaaacataaaatgaatgcaattgttgttgttaacttgtttattgcagcttataatggttacaaataaa
gcaatagcatcacaaatttcacaaataaagcatttttttcactgcattctagttgtggtttgtccaaactcatcaatgtatcttatcatgtctggatct
gcgactctagaggatcataatcagccataccacatttgtagaggttttacttgctttaaaaaacctcccacacctccccctgaacctgaaacat
aaaatgaatgcaattgttgttgttaacttgtttattgcagcttataatggttacaaataaagcaatagcatcacaaatttcacaaataaagcatttttt
tcactgcattctagttgtggtttgtccaaactcatcaatgtatcttatcatgtctggatcccgagcgcgcagccttaattaaGGCTTGTCG
ACGACGGCGGaCTCCGTCGTCAGGATCATtcagacgcggaagcttcgatcaactacgcggacag
pHelper_DBP-IRES-Bxb1 (pHL285):
  (SEQ ID NO: 8)
agaacccctttgatttttaaTGCATcccctctccctcccccccccctaacgttactggccgaagccgcttggaataaggccggtgtgcgtt
tgtctatatgttattttccaccatattgccgtcttttggcaatgtgagggcccggaaacctggccctgtcttcttgacgagcattcctaggggtctt
tcccctctcgccaaaggaatgcaaggtctgttgaatgtcgtgaaggaagcagttcctctggaagcttcttgaagacaaacaacgtctgtagc
gaccctttgcaggcagcggaaccccccacctggcgacaggtgcctctgcggccaaaagccacgtgtataagatacacctgcaaaggcg
gcacaaccccagtgccacgttgtgagttggatagttgtggaaagagtcaaatggctctcctcaagcgtattcaacaaggggctgaaggatg
cccagaaggtaccccattgtatgggatctgatctggggcctcggtacacatgctttacatgtgtttagtcgaggttaaaaaaacgtctaggcc
ccccgaaccacggggacgtggttttcctttgaaaaacacgatgataatatggccacaaccatgccaaaaaagaaaagaaaagtgtatccct
atgatgtccccgattatgccggttcaagagccctggtcgtgattagactgagccgagtgacagacgccaccacaagtcccgagagacagc
tggaatcatgccagcagctctgtgctcagcggggttgggatgtggtcggcgtggcagaggatctggacgtgagcggggccgtcgatcca
ttcgacagaaagaggaggcccaacctggcaagatggctcgctttcgaggaacagccctttgatgtgatcgtcgcctacagagtggaccgg
ctgacccgctcaattcgacatctccagcagctggtgcattgggctgaggaccacaagaaactggtggtcagcgcaacagaagcccacttc
gatactaccacaccttttgccgctgtggtcatcgcactgatgggcactgtggcccagatggagctcgaagctatcaaggagcgaaacagg
agcgcagcccatttcaatattagggccggtaaatacagaggctccctgcccccttggggatatctccctaccagggtggatggggagtgga
gactggtgccagaccccgtccagagagagcggattctggaagtgtaccacagagtggtcgataaccacgaaccactccatctggtggca
cacgacctgaatagacgcggcgtgctctctccaaaggattattttgctcagctgcagggaagagagccacagggaagagaatggagtgct
actgcactgaagagatctatgatcagtgaggctatgctgggttacgcaacactcaatggcaaaactgtccgggacgatgacggagcccctc
tggtgagggctgagcctattctcaccagagagcagctcgaagctctgcgggcagaactggtcaagactagtcgcgccaaacctgccgtg
agcaccccaagcctgctcctgagggtgctgttctgcgccgtctgtggagagccagcatacaagtttgccggcggagggcgcaaacatcc
ccgctatcgatgcaggagcatggggttccctaagcactgtggaaacgggacagtggccatggctgagtgggacgccttttgcgaggaac
aggtgctggatctcctgggtgacgctgagcggctggaaaaagtgtgggtggcaggatctgactccgctgtggagctggcagaagtcaatg
ccgagctcgtggatctgacttccctcatcggatctcctgcatatagagctgggtccccacagagagaagctctggacgcacgaattgctgc
actcgctgctagacaggaggaactggagggcctggaggccaggccctctggatgggagtggcgagaaaccggacagaggtttgggga
ttggtggagggagcaggacaccgcagccaagaacacatggctgagatccatgaatgtccggctcacattcgacgtgcgcggtggcctga
ctcgaaccatcgattttggcgacctgcaggagtatgaacagcacctgagactggggtccgtggtcgaaagactgcacactgggatgtccta
aacccttgccgtctgcgccgt
pTrans/Cis.Rep2-EGFP-Cap2 (pHL300):
(SEQ ID NO: 9)
gttgcgcagccatcgacgtcaGTGGTTTGTCTGGTCAACCACCGCGGaCTCAGTGGTGTACGGTA
CAAACCCAttaattaaggccttaattaggctgcgcgctcgctcgctcactgaggccgcccgggcaaagcccgggcgtcgggcgac
ctttggtcgcccggcctcagtgagcgagcgagcgcgcagagagggagtggccaactccatcactaggggttccttgtagttaatgattaac
ccgccatgctacttatctaccagggtaatggggatcctctagaactatagctagtcgacattgattattgactagttattaatagtaatcaattac
ggggtcattagttcatagcccatatatggagttccgcgttacataacttacggtaaatggcccgcctggctgaccgcccaacgacccccgcc
cattgacgtcaataatgacgtatgttcccatagtaacgccaatagggactttccattgacgtcaatgggtggagtatttacggtaaactgccca
cttggcagtacatcaagtgtatcatatgccaagtacgccccctattgacgtcaatgacggtaaatggcccgcctggcattatgcccagtacat
gaccttatgggactttcctacttggcagtacatctacgtattagtcatcgctattaccatgtcgaggccacgttctgcttcactctccccatctccc
ccccctccccacccccaattttgtatttatttattttttaattattttgtgcagcgatgggggcggggggggggggcgcgcgccaggcggggc
ggggggggcgaggggggggcggggcgaggcggagaggtgcggcggcagccaatcagagcggcgcgctccgaaagtttcctttt
atggcgaggcggcggcggcggcggccctataaaaagcgaagcgcgcggcgggcgggagcaagctttattgcggtagtttatcacagtt
aaattgctaacgcagtcagtgcttctgacacaacagtctcgaacttaagctgcagaagttggtcgtgaggcactgggcaggtaagtatcaag
gttacaagacaggtttaaggagaccaatagaaactgggcttgtcgagacagagaagactcttgcgtttctgataggcacctattggtcttact
gacatccactttgcctttctctccacaggtgtccactcccagttcaattacagctcttaaggctagagtacttaatacgactcactataggctagt
aatacgactcactatagatggtgagcaagggcgaggagctgttcaccggggtggtgcccatcctggtcgagctggacggcgacgtaaac
ggccacaagttcagcgtgtccggcgagggcgagggcgatgccacctacggcaagctgaccctgaagttcatctgcaccaccggcaagc
tgcccgtgccctggcccaccctcgtgaccaccctgacctacggcgtgcagtgcttcagccgctaccccgaccacatgaagcagcacgact
tcttcaagtccgccatgcccgaaggctacgtccaggagcgcaccatcttcttcaaggacgacggcaactacaagacccgcgccgaggtg
aagttcgagggcgacaccctggtgaaccgcatcgagctgaagggcatcgacttcaaggaggacggcaacatcctggggcacaagctgg
agtacaactacaacagccacaacgtctatatcatggccgacaagcagaagaacggcatcaaggtgaacttcaagatccgccacaacatcg
aggacggcagcgtgcagctcgccgaccactaccagcagaacacccccatcggcgacggccccgtgctgctgcccgacaaccactacct
gagcacccagtccgccctgagcaaagaccccaacgagaagcgcgatcacatggtcctgctggagttcgtgaccgccgccgggatcact
ctcggcatggacgagctgtacaagtaaagcggccctagcgtttaaacgggccctctagactcgaggacggggtgaactacgcctgagga
tccgatctttttccctctgccaaaaattatggggacatcatgaagccccttgagcatctgacttctggctaataaaggaaatttattttcattgcaa
tagtgtgttggaattttttgtgtctctcactcggaagcaattcgttgatctgaatttcgaccacccataatacccattaccctggtagataagtagc
atgggggttaatcattaactacaaggaacccctagtgatggagttggccactccctctctgcgcgctcgctcgctcactgaggccgggcg
accaaaggtcgcccgacgcccgggctttgcccgggcggcctcagtgagcgagcgagcgcgcagccttaattaaGGCTTGTCG
ACGACGGCGGaCTCCGTCGTCAGGATCATtcagacgcggaagcttcgatcaactacgcagacag
pAAVS1 LHA-SA-P2A-BxB1-T2A-PuroR-bGH pA-RHA (pHL380):
  (SEQ ID NO: 10)
CTCTCTCCTGAGTCCGGACCACTTTGAGCTCTACTGGCTTCTGCGCCGCCTCTGGCCC
ACTGTTTCCCCTTCCCAGGCAGGTCCTGCTTTCTCTGACCTGCATTCTCTCCCCTGGG
CCTGTGCCGCTTTCTGTCTGCAGCTTGTGGCCTGGGTCACCTCTACGGCTGGCCCAG
ATCCTTCCCTGCCGCCTCCTTCAGGTTCCGTCTTCCTCCACTCCCTCTTCCCCTTGCTC
TCTGCTGTGTTGCTGCCCAAGGATGCTCTTTCCGGAGCACTTCCTTCTCGGCGCTGC
ACCACGTGATGTCCTCTGAGCGGATCCTCCCCGTGTCTGGGTCCTCTCCGGGCATCT
CTCCTCCCTCACCCAACCCCATGCCGTCTTCACTCGCTGGGTTCCCTTTTCCTTCTCC
TTCTGGGGCCTGTGCCATCTCTCGTTTCTTAGGATGGCCTTCTCCGACGGATGTCTCC
CTTGCGTCCCGCCTCCCCTTCTTGTAGGCCTGCATCATCACCGTTTTTCTGGACAACC
CCAAAGTACCCCGTCTCCCTGGCTTTAGCCACCTCTCCATCCTCTTGCTTTCTTTGCC
TGGACACCCCGTTCTCCTGTGGATTCGGGTCACCTCTCACTCCTTTCATTTGGGCAGC
TCCCCTACCCCCCTTACCTCTCTAGTCTGTGCTAGCTCTTCCAGCCCCCTGTCATGGC
ATCTTCCAGGGGTCCGAGAGCTCAGCTAGTCTTCTTCCTCCAACCCGGGCCCCTATG
TCCACTTCAGGACAGCATGTTTGCTGCCTCCAGGGATCCTGTGTCCCCGAGCTGGGA
CCACCTTATATTCCCAGGGCCGGTTAATGTGGCTCTGGTTCTGGGTACTTTTATCTGT
CCCCTCCACCCCACAATCaagcttctgacctcttctcttcctcccacagggcctcgagagatctggcagcggagcaacaaa
cttctcactactcaaacaagcaggtgacgtggaggagaatcccgggcctaggctcgagatgccaaaaaagaaaagaaaagtgtatcccta
tgatgtccccgattatgccggttcaagagccctggtcgtgattagactgagccgagtgacagacgccaccacaagtcccgagagacagct
ggaatcatgccagcagctctgtgctcagcggggttgggatgtggtcggcgtggcagaggatctggacgtgagcggggccgtcgatccat
tcgacagaaagaggaggcccaacctggcaagatggctcgctttcgaggaacagccctttgatgtgatcgtcgcctacagagtggaccgg
ctgacccgctcaattcgacatctccagcagctggtgcattgggctgaggaccacaagaaactggtggtcagcgcaacagaagcccacttc
gatactaccacaccttttgccgctgtggtcatcgcactgatgggcactgtggcccagatggagctcgaagctatcaaggagcgaaacagg
agcgcagcccatttcaatattagggccggtaaatacagaggctccctgcccccttggggatatctccctaccagggtggatggggagtgga
gactggtgccagaccccgtccagagagagcggattctggaagtgtaccacagagtggtcgataaccacgaaccactccatctggtggca
cacgacctgaatagacgcggcgtgctctctccaaaggattattttgctcagctgcagggaagagagccacagggaagagaatggagtgct
actgcactgaagagatctatgatcagtgaggctatgctgggttacgcaacactcaatggcaaaactgtccgggacgatgacggagcccctc
tggtgagggctgagcctattctcaccagagagcagctcgaagctctgcgggcagaactggtcaagactagtcgcgccaaacctgccgtg
agcaccccaagcctgctcctgagggtgctgttctgcgccgtctgtggagagccagcatacaagtttgccggcggagggcgcaaacatcc
ccgctatcgatgcaggagcatggggttccctaagcactgtggaaacgggacagtggccatggctgagtgggacgccttttgcgaggaac
aggtgctggatctcctgggtgacgctgagcggctggaaaaagtgtgggtggcaggatctgactccgctgtggagctggcagaagtcaatg
ccgagctcgtggatctgacttccctcatcggatctcctgcatatagagctgggtccccacagagagaagctctggacgcacgaattgctgc
actcgctgctagacaggaggaactggagggcctggaggccaggccctctggatgggagtggcgagaaaccggacagaggtttgggga
ttggtggagggagcaggacaccgcagccaagaacacatggctgagatccatgaatgtccggctcacattcgacgtgcgcggtggcctga
ctcgaaccatcgattttggcgacctgcaggagtatgaacagcacctgagactggggtccgtggtcgaaagactgcacactgggatgtccG
GATCCGGTGAGGGCAGAGGAAGTCTACTAACATGCGGTGACGTGGAGGAGAATCC
GGGCCCTGAATTCGCCACCATGACAGAATACAAGCCCACAGTCAGGCTTGCAACTA
GAGATGACGTTCCCAGAGCAGTGAGAACCCTGGCAGCTGCTTTTGCAGACTATCCG
GCCACGAGGCACACTGTCGATCCCGATCGGCACATCGAGCGCGTTACAGAATTGCA
GGAACTGTTCCTGACAAGAGTTGGGCTCGACATTGGTAAAGTGTGGGTCGCTGACG
ACGGGGCAGCTGTTGCGGTGTGGACCACACCGGAGAGTGTGGAGGCCGGTGCTGTG
TTTGCCGAAATTGGTCCACGCATGGCCGAACTCTCCGGATCTCGGTTGGCCGCACAG
CAGCAGATGGAAGGCCTGCTGGCGCCTCACCGACCTAAAGAGCCTGCATGGTTTCT
GGCCACCGTCGGCGTATCCCCCGATCATCAGGGTAAGGGCCTCGGCAGCGCCGTCG
TGCTGCCGGGTGTTGAGGCAGCTGAAAGAGCAGGCGTGCCGGCGTTTTTGGAAACA
AGTGCACCGAGGAATCTCCCATTTTACGAGAGACTGGGGTTCACCGTGACAGCCGA
TGTCGAAGTGCCCGAAGGCCCCAGGACCTGGTGTATGACCCGCAAGCCCGGTGCCT
AGgatccgatctttttccctctgccaaaaattatggggacatcatgaagccccttgagcatctgacttctggctaataaaggaaatttattttca
ttgcaatagtgtgttggaattttttgtgtctctcactcgTcccgatcccctatggtcgactctcagtacaatctgctctgatgccgcatagttaag
ccagtatctgctccctgcttgtgtgttggaggtcgctgagtagtgcgcgagcaaaatttaagctacaacaaggcaaggcttgaccgacaatt
gcatgaagaatctgcttagggttaggcgttttgcgctgcttcgcgatgtacgggccagatatacgcgtGCCACTAGGGACAGG
ATTGGTGACAGAAAAGCCCCATCCTTAGGCCTCCTCCTTCCTAGTCTCCTGATATTG
GGTCTAACCCCCACCTCCTGTTAGGCAGATTCCTTATCTGGTGACACACCCCCATTT
CCTGGAGCCATCTCTCTCCTTGCCAGAACCTCTAAGGTTTGCTTACGATGGAGCCAG
AGAGGATCCTGGGAGGGAGAGCTTGGCAggggggggagggaagggggggaTGCGTGACCTGC
CCGGTTCTCAGTGGCCACCCTGCGCTACCCTCTCCCAGAACCTGAGCTGCTCTGACG
CGGCCGTCTGGTGCGTTTCACTGATCCTGGTGCTGCAGCTTCCTTACACTTCCCAAG
AGGAGAAgcagtttggaaaaacaaaatcagaataagttggtcctgagttctaactttggctcttcacctttctagtccccaatttatattgt
tcctccgtgcgtcagttttacctgtgagataaggccagtagccagccccgtcctggcagggctgtggtgaggaGGGGGGTGTCC
GTGTGGAAAACTCCCTTTGTGAGAATGGTGCGTCCTAGGTGTTCACCAGGTCGTGGC
CGCCTCTACTCCCTTTCTCTTTCTCCATCCTTCTTTCCTTAAAGAGTCCCCAGTGCTAT
CTGGGACATATTCCTCCGCCCAGAGCAGGGTCCCGCTTCCCTAAGGCCCTGCTCTGG
GCTTCTGGGTTTGAGTCCTTGGCAAGCCCAGGAGAGGCGCTCAGGCTTCCCTGTCCC
CCTTCCTCGTCCACCATCTCATGCCCCTGGCTCTCCTGCCCCTTCCCTACAGGGGTTC
CTGGCTCTGCTCTTCAGAC
aTTR:
  (SEQ ID NO: 11)
GTGGTTTGTCTGGTCAACCACCGCGGaCTCCGTCGTCAGGATCAT
```

1. A circular nucleic acid comprising:
(i) a first nucleic acid sequence encoding one or more adenoviral helper factors; and
(ii) a second nucleic acid sequence encoding a recombinase.

2. The circular nucleic acid of claim 1, wherein the circular nucleic acid comprises a plasmid.

3. The circular nucleic acid of claim 1, wherein the circular nucleic acid comprises a minicircle.

4. The circular nucleic acid of claim 1, wherein:
(i) the first nucleic acid sequence encoding the one or more adenoviral helper factors is positioned 5′ relative to the nucleic acid sequence encoding the recombinase,
(ii) the one or more adenoviral helper factors comprise E2A, E1B55K, E4orf6, and/or VA RNA transcription units,
(iii) the recombinase is a Bxb1 recombinase, and/or
(iv) the second nucleic acid sequence encoding the recombinase is inserted before a stop codon of the nucleic acid sequence encoding the one or more adenoviral helper factors.

5.-7. (canceled)

8. The circular nucleic acid of claim 4, wherein the stop codon is a stop codon of E2A helper factor.

9. A circular nucleic acid comprising:
(i) a first nucleic acid sequence encoding an adeno-associated virus (AAV) Rep;
(ii) a second nucleic acid sequence encoding a transgene comprising a promoter operably linked to a nucleic acid sequence encoding a gene product, flanked by AAV inverted terminal repeats (ITRs); and
(iii) a third nucleic acid sequence encoding an AAV capsid protein.

10. The circular nucleic acid of claim 9, wherein the circular nucleic acid comprises a plasmid or wherein the circular nucleic acid comprises a minicircle.

11. (canceled)

12. The circular nucleic acid of claim 9, further comprising a first recombinase recognition site positioned between the first nucleic acid sequence and the second nucleic acid sequence; and a second recombinase recognition site positioned between the second nucleic acid sequence and the third nucleic acid sequence.

13. The circular nucleic acid of claim 12, wherein the first recombinase recognition site comprises an attP site and the second recombinase recognition site comprises an attB site.

14. The circular nucleic acid of claim 9, wherein the second nucleic acid sequence is positioned between a p40 promoter of the first nucleic acid sequence and an intron of the third nucleic acid sequence.

15. The circular nucleic acid of claim 14, wherein the intron of the third nucleic acid sequence is positioned upstream of a start codon of the nucleic acid sequence encoding the AAV capsid protein.

16. The circular nucleic acid of claim 9, wherein the first nucleic acid sequence and second nucleic acid sequence are in a first open reading frame, and the third nucleic acid sequence is in a second open reading frame.

17. A circular nucleic acid comprising a transgene comprising a promoter operably linked to a nucleic acid sequence encoding a gene product, flanked by AAV inverted terminal repeats (ITRs), and an attL recombinase recognition site positioned between the AAV ITRs.

18. The circular nucleic acid of claim 17, wherein the circular nucleic acid lacks bacteria-derived DNA.

19. A method for producing adeno-associated virus (rAAV) particles the method comprising introducing into a host cell:
(i) the circular nucleic acid of claim 1; and
(ii) a second circular nucleic acid comprising a first nucleic acid sequence encoding an adeno-associated virus (AAV) Rep; a second nucleic acid sequence encoding a transgene comprising a promoter operably linked to a nucleic acid sequence encoding a gene product, flanked by AAV inverted terminal repeats (ITRs); and a third nucleic acid sequence encoding an AAV capsid protein; and
culturing the host cell under conditions under which rAAV particles are produced.

20. The method of claim 19, wherein less than 1% of the rAAV particles comprise plasmid backbone DNA.

21. The method of claim 19, further comprising
a third circular nucleic acid encoding one or more adenoviral helper factors; and culturing the host cell under conditions under which rAAV particles are produced.

22. The method of claim 21, wherein the adenoviral helper factors comprise E2A, E4orf6, and/or VA RNA transcription units.

23. (canceled)

24. A host cell, wherein a recombinase is integrated into the genome of the host cell, optionally wherein the recombinase is Bxb1,
wherein the recombinase is integrated into a AAVS1 safe harbor locus of the host cell, and/or
wherein the host cell is a HEK293 cell.

25.-27. (canceled)

28. A method for producing adeno-associated virus (rAAV) particles the method comprising administering to the host cell of claim 24:
(i) the circular nucleic acid comprising a first nucleic acid sequence encoding an adeno-associated virus (AAV) Rep; a second nucleic acid sequence encoding a transgene comprising a promoter operably linked to a nucleic acid sequence encoding a gene product, flanked by AAV inverted terminal repeats (ITRs); and a third nucleic acid sequence encoding an AAV capsid protein; and
(ii) a second circular nucleic acid encoding one or more adenoviral helper factors;
wherein the host cell is cultured under conditions under which rAAV particles are produced.

29.-30. (canceled)
